(19) United States
(12) Patent Application Publication
(10) Pub. No.: US 2009/0098017 A1
(43) Pub. Date: Apr. 16, 2009

(54) NANOPOROUS MEMBRANE EXCHANGER

(75) Inventors: Zeynep Celik-Butler, Colleyville, TX (US); Robert C. Eberhart, Dallas, TX (US); Richard E. Billo, Colleyville, TX (US); Cheng-Jen Chuong, Arlington, TX (US); Richard B. Timmons, Arlington, TX (US); Vijayakrishnan Ambravaneswaran, Arlington, TX (US)

Correspondence Address: ROSENBAUM & ASSOCIATES, P.C., 650 DUNDEE ROAD, SUITE #380, NORTHBROOK, IL, 60062, US

(73) Assignee: Board of Regents, The University of Texas System, Austin, TX (US)

(21) Appl. No.: 11/873,142

(22) Filed: Oct. 16, 2007

(51) Int. Cl. A61M 1/00

(52) U.S. Cl. 422/48

(57) ABSTRACT

The invention is a nanoporous membrane exchanger.

### BACKGROUND

The invention generally relates to nanoporous membranes, and more particularly relates to mass exchanger systems.

Mass exchangers used in medical devices include kidney dialysis, plasmapheresis machines, drug delivery systems, and oxygen mass exchangers or oxygenators. The oxygenator is a gas exchange system that serves to enrich the blood with oxygen and remove carbon dioxide. Oxygenators serve as a key component of heart-lung machines for open-heart surgery and extracorporeal life support. Most current oxygenator designs interpose an open pore polymeric membrane between the gas and blood channels. These so-called membrane oxygenators suffer from inefficient gas exchange; in particular, the inability to match the highly efficient transfer of oxygen and carbon dioxide made possible by capillary blood channels with diameters only slightly larger than red cell dimensions. Furthermore, current membrane oxygenators are considered to be responsible for the post-perfusion complications of open heart surgery, e.g., thrombosis, embolization, and activation of inflammatory pathways, owing to a combination of blood flow disturbances and incompatible polymers. Thus the successful fabrication of extracorporeal and implantable miniature blood oxygenators with improved nanoporous membranes can benefit many thousands of patients undergoing heart lung bypass, additional large numbers of patients with respiratory failure of diverse etiologies, and patients requiring a bridge to lung transplantation, and permanent lung implantation.

Current microporous membranes are of relatively large size, with dimensions that make it impossible to control blood channel dimensions at the scale of the pulmonary capillaries. Moreover, the control over the pore size is poor due to the undiscriminating techniques used in microfabrication. The standard deviation of the pore size distribution and the nonuniform spatial placement of the pores deteriorate even further with decreasing pore diameter. The ability to precisely control the feature size topography and the surface chemistry of the pores will make possible the development of a small, efficient blood oxygenator that was not within reach previously. Current microporous membranes cannot be effectively used for extended periods of time, for example in longer term pulmonary support procedures. The dimensions of the micropores of the microporous membranes are so large that blood plasma can penetrate from the blood side of the membrane to the gas side, blocking the pores and thereby substantially reducing gas exchange efficiency. Furthermore, lipoproteins contained in the blood plasma adsorb to the pore channel walls, lowering the surface tension that had supported the exclusion of plasma from the micropores, thereby converting these channels into hydrophilic conduits. The micropores then permit transport of copious amounts of water and plasma constituents from the blood to the gas space, creating a pulmonary edema that shuts down the gas exchange process and requires prompt and repeated replacement of the oxygenator. The present invention attempts to solve these problems, as well as others.

### SUMMARY OF THE INVENTION

Provided herein are systems, methods and compositions for a nanoporous membrane exchanger.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1A is a perspective view of the cross-section of the dome channel design.

FIG. 2A is a cross-sectional view of the silicon layer and the silicon nitride layer; FIG. 2B is a cross-sectional view of the fast etch along <100>; FIG. 2C is a cross-sectional view of the slow etch along the <111>; FIG. 2D is a cross-sectional view of the removal of the silicon nitride and the focused ion beam drilling of the nanopores.

FIG. 3 is a schematic of the geometric position of the nanoporous channel during focused ion beam (“FIB”) using a FIB system.

FIG. 4A is a Scanning Electron Microscope SEM image of the array of nanopores with a 4 μm diameter; FIG. 4B is an SEM image of the array of nanopores on the nanoporous membrane; FIG. 4C is an SEM image to show the depth of the nanopores; and FIG. 4D is an SEM image of 10 nm holes nanoimprinted on a resist material.

FIG. 5 is a schematic diagram showing a Radiofrequency (“RF”) plasma discharge system.

FIG. 6 is a graph showing data from the toe region of all the stress-strain data

FIG. 7 is a graph showing the comparison of porous and non-porous membrane for variation of pressure with respect to the membrane displacement.

FIG. 8 is a schematic diagram showing the test chamber of the oxygen permeation analyzer.

FIG. 9 is a cross-section schematic of the membrane, steel plate, masking foil, and operational parameters.

FIG. 10A is perspective view of the dome channel design; FIG. 10B is a cross-section of the dome channel.

FIG. 11 is a graph of the variation of the blood to gas column with the width of the gas channel for different heights of the blood channel.

FIG. 12A is a perspective view of the roof-top/dome channel design; FIG. 12B is a cross-section of the roof-top/dome channel.

FIG. 13A is a graph of the variation of the blood to gas ratio with respect to the width of the gas channel; FIG. 13B is a graph of the ratio of the interaction surface area to blood volume vs. the width of the gas channel.

FIG. 14A is a perspective view of the roof top channel design 400; FIG. 14B is a cross-section of the roof top channel 410.

FIG. 15A is a perspective view of the roof top channel design 500; FIG. 15B is a cross-section of the roof top channel 510.

FIG. 16 is graph comparing roof top channel design 400 and roof top channel design 500 for variation in blood to gas volume ratio as a function of the gas channel width.

FIG. 17A is the steady state velocity distribution across the blood channel for the roof-top dome channel when perfused under pressure gradient of 36 cm H₂O; and FIG. 17B is the velocity profile along the vertical center line in blood channel.

FIG. 18A is the dome channel design with side channels connected to gas manifold, shown as yellow rectangles on the side; FIG. 18B is the deflection of the nanoporous membrane under pressure load from the blood channel; and FIG. 18C is the Von Mises stress distribution on the nanoporous membrane due to blood channel pressurization.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

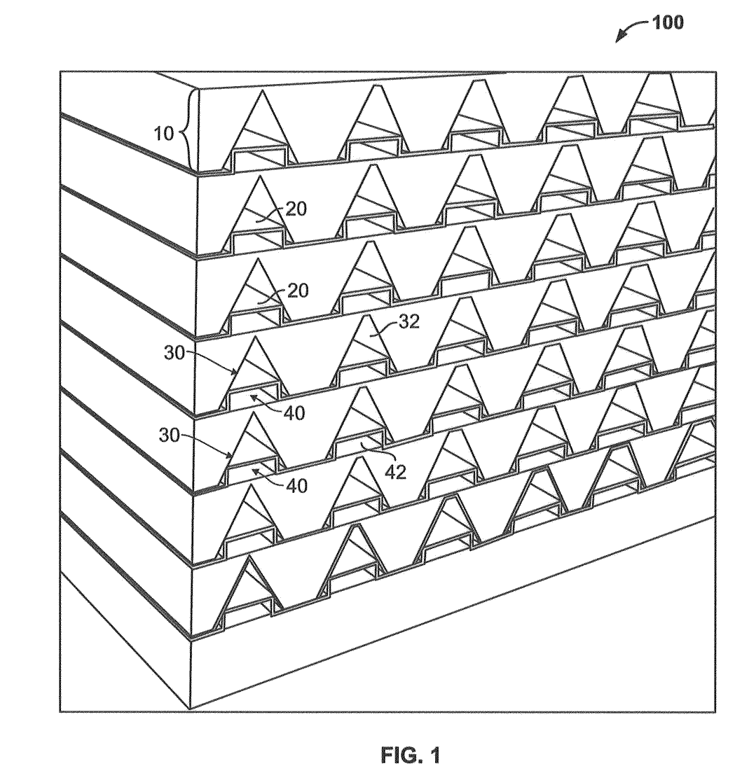

Generally speaking, the nanoporous membrane exchanger 100 comprises a plurality of nanoporous channels 10, as shown in FIG. 1. The nanoporous channels 10 include a nanoporous membrane 20, a gas channel 30, and a blood channel 40. The nanoporous membrane includes a plurality of nanopores 22. The nanoporous membrane exchanger 100 includes several nanoporous channel designs, which include, but are not limited to, a dome channel design 200, a roof-top dome channel design 300, a roof-top channel design 400, and a roof-top channel design 500. Other channel designs will be apparent to those skilled in the art. Such nanoporous channel designs can be combined and/or varied as to produce the nanoporous membrane blood exchanger 100 with optimum size, strength, and/or smart capabilities.

It should be appreciated that the nanoporous membrane exchanger 100 includes all classes of mass exchangers used in medical devices, including oxygenator mass exchangers that can function with nanopores 22 in blood channels 40 approximating blood capillary dimensions, and kidney dialysis and plasmapheresis machines, drug delivery systems, and the like. By way of example only, the various embodiments and examples of the nanoporous membrane exchanger 100 are detailed according to an oxygenator mass exchanger; however, it will be understood that the invention is capable of further modifications for kidney dialysis, plasmapheresis, and drug delivery.

“Blood to gas volume ratio” is the ratio of blood volume per unit length to gas volume per unit length. “Surface area of interaction” is the gas exchange surface area of between the blood and gas channels. “Membrane” is a suspended structure formed by etching of the substrate. “Porosity” is the ratio of nanopore volume to membrane volume, otherwise the ratio of total nanopore surface area to membrane area. “Pitch” is the distance between the centers of two adjacent nanopores.

The use of brackets ‘[ ]’ herein in conjunction with such numbers as ‘111’ and ‘110’ pertains to a direction or orientation of a crystal lattice and is intended to include directions ‘< >’ within its scope, for simplicity herein. The use of parenthesis ‘( )’ herein with respect to such numbers ‘11’ and ‘110’ pertains to a plane or a planar surface of a crystal lattice and is intended to include planes ‘{ }’ within its scope for simplicity herein. Such use is intended to follow common crystallographic nomenclature known in the art.

In one embodiment of the invention, the nanopores 22 permit the oxygenation of blood 42 takes place between the blood channel 40 and the gas channel 30. For example, gas 32 processes through the gas channel 30 and blood 42 processes through the blood channel 40. Alternatively, other mass may transfer through the gas channel that is not a gas, and other mass may transfer through the blood channel that is not blood. For example, a drug composition may travel through the gas channel to permit the exchange of drugs to the blood channel. The blood channels 40 are narrow to give blood cells direct access to the nanoporous membrane and achieve a high-efficiency of oxygenation with the gas channel 30. The nanoporous membrane 20 includes a mechanical strength to withstand the flow of blood and low internal stress to be free-standing with no deformation. In one embodiment of the invention, the nanoporous membrane 20 can include multi-compartmental structures, nanoscale ridges to entrain adsorbed proteins into innocuous channels, multi-level multi-size pre-structure for immobilization of certain molecules, and biosensors can be added to the nanoporous channels. The thickness of the nanoporous membrane 20 allows for the maximum diffusion rate, approximately 500 nm in one embodiment of the invention. The size, location, and shape of the nanopores are individually controllable. The shape of the nanopore 22 is straight-through for a high diffusion rate. The nanoporous channels 10 are biocompatible and the small nanopore 22 size prevents host defense activation.

In one embodiment of the invention, the gas exchange efficiency of the nanoporous membrane exchanger 100 closely matches the gas exchange efficiency of the natural human lung. The capillaries in the natural lung includes a surface area of 70 m², a blood path width of 8 μm, a blood path length of 200 μm, a membrane thickness of 0.5 μm, and a maximum oxygen transfer of 2000 ml/min STP, i.e. Standard Temperature and Pressure. Red blood cells undergo shape deformation when transiting through the capillaries for efficient gas exchange, where the red blood cells undergo a torpedo-to-parachute shape to substantially increase oxygenation efficiency. A red blood cell deforms to a torpedo-like-shape in a capillary approximately 4 μm in diameter and a red blood cell deforms to a parachute-like-shape in a capillary approximately 7 μm in diameter. The nanoporous membrane exchanger 100 includes channels 40 with a diameter and membranes 20 with a mechanical strength to permit withstanding the torpedo-to-parachute shape deformation of the red blood cell for oxygenation efficiency. The ratio of the surface area of interaction to blood volume is balanced in the membrane exchanger 100 to obtain an efficient gas exchange rate. The membrane exchanger 100 also maintains an acid-base balance. The surface area of interaction of blood-gas in the nanoporous membrane exchanger 100 increases blood oxygenation. The nanoporous membrane 20 includes a precisely controlled porosity, where the dimensions of the nanopores 22 are drilled in a controlled fashion. The placement of the nanopores 22 is controlled to obtain the required porosity. The nanoporous membrane 20 withstands pressure exerted by gas and blood during the exchange of gases from either side of the nanoporous membrane. Precise control of the feature size, number density, chemistry and topography of the nanopores 22 allows for addition of gas sensors with accurate separation and selectivity, functional cell-sorting, protein patterning and blood exchanger membranes and other mass transfer membranes, such as plasmaphoresis, drug delivery for short and long term treatments.

### Fabrication of Nanoporous Channels

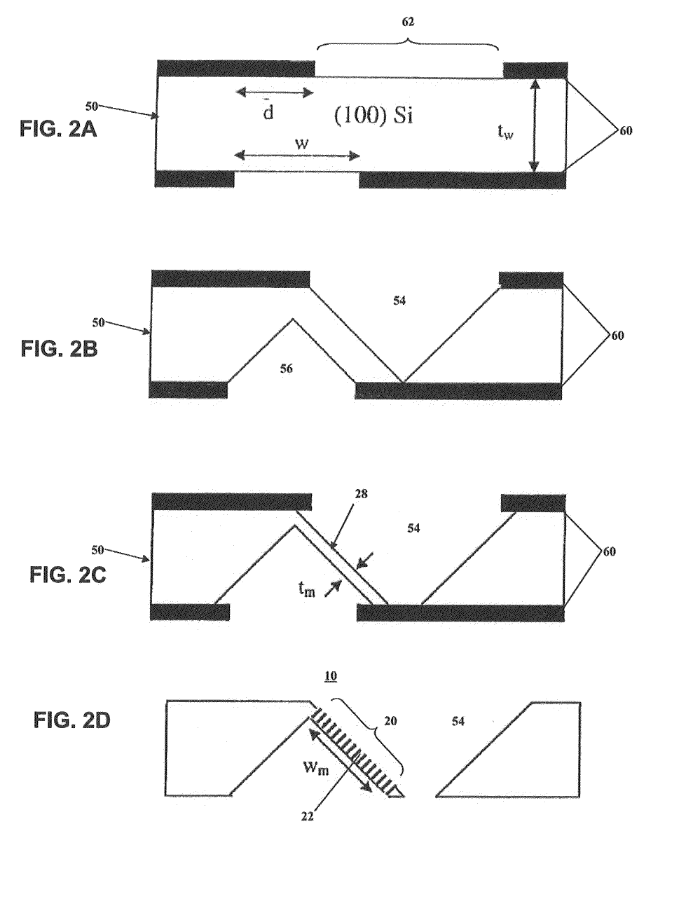

In another embodiment of the invention, the nanoporous channel 10 is fabricated from a layer of silicon 50 and a layer of silicon nitride 60 (“Si₃N₄”), as shown in FIG. 2A. Alternatively, the nanoporous channel 10 may include a layer of silicon carbide, silicon oxide, gallium nitride, and the like for the nanoporous membrane 20. The nanoporous channel 10 includes a depth d, a width w, and a thickness of the silicon wafer 50 t_{w}. The width w is in the <100> direction of the silicon layer 50. The first step is a standard deposition of silicon nitride 60 on the front and back of the (100) surfaces of the silicon wafer 50 using Chemical Vapor Deposition defined by common photolithography. In one embodiment of the invention, Low Pressure Chemical Vapor Deposited (“LP-CVD”) of silicon nitride 60 fabricates the nanoporous membrane 20. Alternatively, other deposition techniques may deposit the silicon nitride, i.e. ultrahigh vacuum CVD, plasma enhanced CVD, aerosol assisted CVD, atomic layer CVD, and the like. Etching the windows such that an opening 62 at the back surface of the silicon wafer 50 is obtained as stripes along the wafer, as shown in FIG. 2A. Etching is used in microfabrication to chemically remove layers from the surface of a wafer during manufacturing. In one embodiment of the invention, SU-8 2010 (MicroChem Corp, Newton, Mass.) protects the silicon nitride 30 during the etching process. SU-8 2010 is a high contrast epoxy based photoresist for micromachining. Then, in one embodiment of the invention, a fast anisotropic wet etch such as by Potassium Hydroxide (“KOH”) or Ethylene Diamine Pyrocatechol (“EDP”) is used to etch along the <100> direction, as shown in FIG. 2B. Anisotropic wet etching uses wet etchants to etch crystalline materials at very different rates depending upon which crystal face is exposed. KOH can achieve selectivity of 400 between <100> and <111> planes. EDP (an aqueous solution of ethylene diamine and pyrocatechol), which also displays high selectivity for p-type doping. Tetramethylammonium hydroxide (“TMAH”), CsOH, NaOH, and N₂H₄—H20 are also other options for anisotropic wet etching.

The fast anisotropic wet etch rate in the <100> direction is about 1-2 μm/min, depending on the dilution, and may take place at roughly 1.5 nm/min. The fast etch exposes the (111) planes and forms channels 54 and 56 in the silicon layer 50. Then, a very slow etching in the <111> direction by KOH etching creates the membrane 28 with thickness tₘ, as shown in FIG. 2C. The slow etch proceeds very slowly in the <111> direction, roughly 2-5 nm/min, allowing precise control of the membrane thickness tₘ oriented on the (111) plane. Since the anisotropic etch angle between <111> and <100> is 54.7 degrees, the thickness, tₘ of the resultant membrane:

$${t}_{m} = ( d + \frac{{t}_{w}}{tan 54.7 °} - w - rt ) cos 54.7 ° \qquad ( 1 )$$

Here, t_{w} is the silicon wafer 50 thickness, w is the width of the channel and d is the distance offset between the windows on the front and back silicon wafer 20 surfaces, as shown in FIG. 1A. The etch rate in <111> is r; the total etching time is t. The resultant membrane width can be expressed as:

$${w}_{m} = \frac{w}{2 cos 54.7 °} + \frac{rt}{sin 54.7 °cos 54.7 °} \qquad ( 2 )$$

This method is effective in obtaining membranes 28 down to 770 nm or lower. Thinner membranes can be achieved by increasing the etch rate and/or thinning the silicon wafer 50 prior to starting process. Thinning the silicon wafer 50 would also decrease the volume of the channel 54, as shown in FIG. 2D. Typical 4″ silicon wafers are 400-600 μm thick; however, silicon wafers thinned down to 30-100 μm are also suitable. In one embodiment of the invention, the silicon nitride layer 30 is removed and Focused Ion Beam (“FIB”) drilling creates the nanopores 22 of the nanoporous membrane 20.

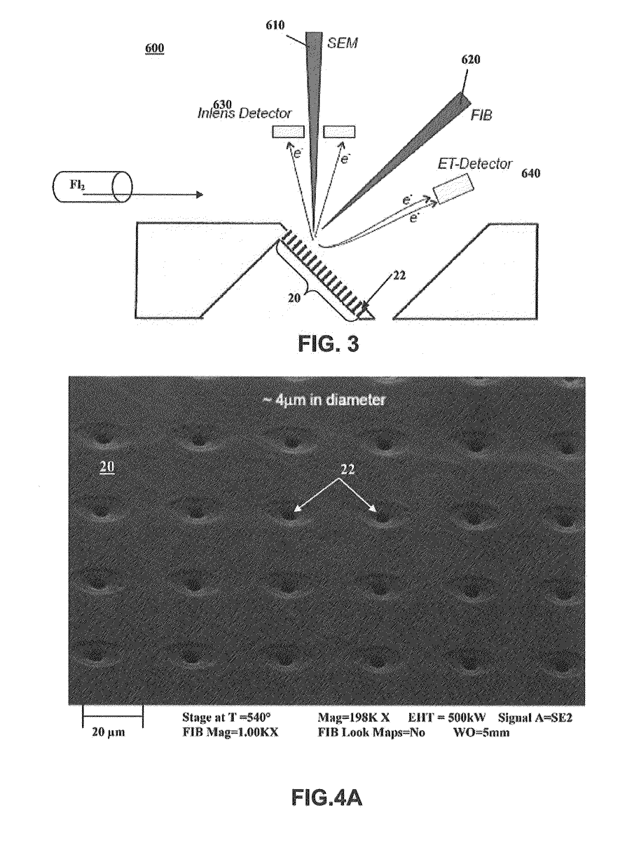

In one embodiment of the invention, the nanopores 22 are drilled through the membrane 28 in a high vacuum chamber using FIB assisted with injected fluorine gas and coating the membrane 28 with a thin layer of gold, as shown in FIG. 2D. The thickness of the gold layer may be approximately 10 nm, which is to reduce the charging effect caused by the gallium ions (Ga⁺) when drilling the pores 22. Gold sputtered on the membrane side to reduce charging. FIG. 3 shows the Zeiss Cross-Beam system 600 with Scanning Electron Microscopy 610 (“SEM”) and FIB 620. The use of fluorine gas injection in conjunction with the Ga⁺ ion beam makes the drilling a physical and chemical process. This technique allows drilling of holes in a pattern of 10's of nanometer size with minimal debris and no Ga⁺ remains on the finished membrane. The Zeiss 1540XB CrossBeam® work station 600 (Carl Zeiss, Peabody, Mass.) enables live SEM 610 imaging during FIB 620 operation with automatic end-point detection for drilling, as shown in FIG. 3. The holes are drilled one-by-one in an automated process with the computer controlled stage and the Nabity Pattern Generation System supplied with the workstation. Once the current from the FIB gun is stabilized, the pattern of nanopores 22 is fed into the computer which controls the FIB. The control of the FIB gun uses the external pattern, the system 600 makes nanopores of accurate dimensions and the placement of nanopores is also controlled. FIG. 3 shows the geometrical position of the nanoporous membrane 20 and the detectors during ion drilling. The in-lens detector 630 is located in front of the sample and records information about the sample surface. The Everhart-Tholey detector 640 (“ET detector”) is located behind the nanoporous membrane 20 and records secondary electrons emitted from the backside of the sample to allow precise control of the holes. The SEM operates with a resolution of 1.1 nm @ 20 kV, and the FIB operates with a resolution of 7 nm @30 kV.

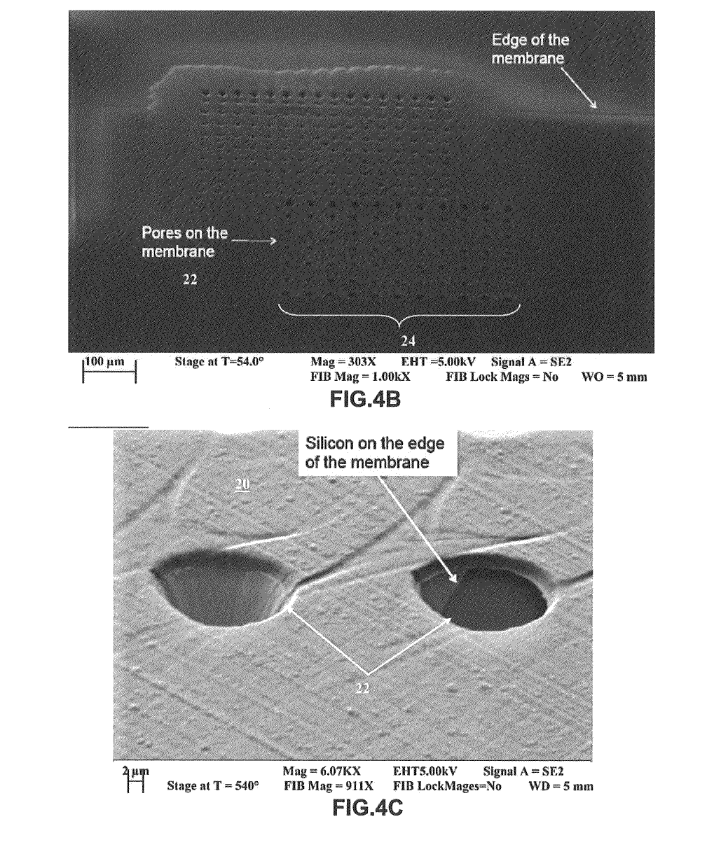

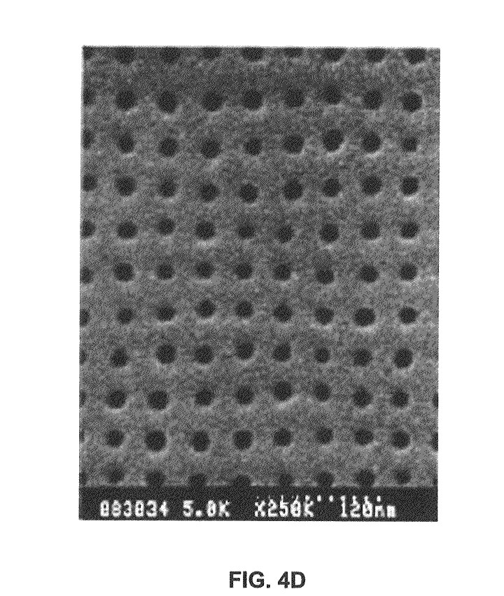

The precisely controlled holes of 4 μm in diameter include an estimated aspect ratio of 1:5, as shown in FIG. 4A. The SEM showing of an array of nanopores 22 holes drilled in membrane 20 using fluorine-gas assisted Ga⁺ ion in the FIB system 600, where a specific pattern 24 of the nanopores 22 is drilled in the membrane 28, as shown in FIG. 4B. Optimization of gas injection rate and the ion dose would allow drilling of smaller holes with higher aspect ratios. There is minimal risk of breaking of chemical bonds in the silicon layer 50 due to loss of energy to the material from the Ga⁺ ion beam. This is inconsequential since the anisotropic etching is done before the drilling. FIG. 4C shows that the drilling of the nanopores 22 has gone all through the membrane 20. The plane of silicon on the edge of the membrane is visible, which was made during the etching of the silicon layer 50. The nanopore 22 on the left side of FIG. 4C includes silicon not etched to form the membrane 20. FIG. 4C confirms that the nanopores 22 have gone through the membrane 20 to result in a nanoporous membrane 20.

In one embodiment of the invention, the nanopore 22 diameter size is in the range of approximately 50-500 nm, and the nanoporous membrane 20 thickness is in the range between 500 nm-5 μm. The porosity is in the range between 0-30 percent. In one embodiment of the invention, the mechanical strength, in three point bending test, has a stiffness ˜1.0 μg/nm. Biocompatibility includes platelet and leukocyte adhesion is less than 10 cells/μm² to avoid thrombosis and immune system activation; fibrinogen and gamma globulin adsorption is less than 3 ng/μm² to avoid protein denaturation-induced activation of host defense systems, including thrombosis and the immune system.

In another embodiment of the invention, the nanopores 22 are generated with a nanoimprinter. A nanoimprinter fabricates nanometer scale patterns and creates patterns by mechanical deformation of imprint resist and subsequent processes (NXB200, Nanonex, N.J.). The imprint resist is typically a monomer or polymer formulation that is cured by heat or UV light during the imprinting. Adhesion between the resist and the template is controlled to allow proper release. Thermoplastic nanoimprint lithography, photo-nanoimprint lithography, nanoscale contact printing, Step-and-Flash nanoimprinting, electrochemical nanoimprinting, and combined nanoimprint and photolithography can be used. The NXB200 conduct all forms of nanoimprinting, including thermoplastic, UV-curable, thermal curable and direct nanoimprinting (embossing). The NXB200 is high throughput large-area patterning of 3D nanostructures with sub-10 nm resolution and accurate overlay alignment for larger membranes than 1 mm². As shown in FIG. 4D, 10 nm diameter nanopore 22 holes are imprinted on a resist material for subsequent lift-off process. Such a process is adapted to nanoimprint nanopores on the silicon nitride membrane. For example, a nanoimprint stamp consisting of regular arrays of Si₃N₄ pyramids may prepare the nanopores. Alternatively, a polymer template, which has an array of nanometer diameter pillar patterns, is fabricated by hot embossing method using anodic aluminum oxide (AAO) template as an embossing stamp. After depositing the thin layer of silicon oxide and coating of anti-adhesion monolayer of organic film on silicon oxide, UV nanoimprint lithography was carried out with the polymer template. As a result, nano-pore array pattern, identical to anodic aluminum oxide pattern, is fabricated on silicon substrate. Residual layer of imprinted nano-pore array pattern is removed by oxygen plasma etch and thin film of Au/Ti was deposited. After lift-off process, Au/Ti dot array was also fabricated on silicon substrate.

The nanoporous channel 10 is precisely aligned and bonded to produce the gas channel 30 and the blood channel 40. The alignment and wafer bonding of the nanoporous channels 10 is repeated laterally along the silicon wafer and vertically by stacking the nanoporous channels 10, as shown in FIG. 1, to produce a nanoporous membrane exchanger of 100's of parallel channels. Wafer-to-wafer alignment using infrared light allows a real-time control loop for the alignment process. The silicon nanoporous channels gets transparent for wavelengths above 1050 nm. Aligned wafer bonding is a wafer-to-wafer 3-D interconnect technology where the wafers are aligned and bonded face to face or back to face, and then thinned and interconnected prior to additional stacking processes or dicing. Wafer bonding and wafer-to-wafer alignment are well established technologies from MEMS manufacturing, but they require processes and equipment enhanced to provide the compatibility with back-end wafer processing, as well as micron-size through-die interconnectivity needed in 3-D ICs. Biocompatible bonding materials such poly(propyl-methacrylate) (“PPMA”) and poly(ethyl-butyl-methacrylate) (“PEBMA”) and other biocompatible materials with high adhesive strength are suitable for bonding the nanoporous channels together. The alignment and bonding of the nanoporous channels includes an accuracy to precisely align for the gas channels and blood channels.

### Membrane Surface Treatment

The nanoporous membrane 20 can include functionalized surface treatments for specific applications without any degradation in the nanoporous membrane properties with chemically inert materials comprising the nanoporous membrane. In one embodiment of the invention, C₁₈ alkylation of a conformal monomolecular nanoporous membrane 20 of ally alcohol to permit albumin adsorption from the whole blood. Serum albumin, the dominant protein in blood, is a “bystander” molecule in respect to the body's host defense systems (thrombus formation, activation of the immune system by various pathways, inflammation, fibrinolysis). Adsorption of the patient's own albumin for coverage of the foreign surfaces prevents the signaling of the host defense systems that activate these responses, due to albumin intrinsic ability to bind molecules.

In one embodiment of the invention, gas phase deposition coats the membrane for blood compatibility to provide uniform coating compositions. Gas phase deposition means by any method whereby the gaseous monomers are attached to the solid substrate as a surface coating. Gas phase depositions include plasma and photochemical induced polymerizations. Plasma induced polymerizations or plasma depositions are polymerizations due to the generations of free radicals caused by passing an electrical discharge through a gas. The electrical discharge can be caused by high voltage methods, either alternating current (“AC”) or direct current (“DC”), or by electromagnetic methods, such as, radio frequency (“RF”) and microwave. Alternatively, the coating process can be carried out using photochemical induced polymerizations as provided by direct absorption of photons of sufficient energy to create free radicals and/or electronically excited species capable of initiation of the polymerization process.

In one embodiment of the invention, radio frequency plasma polymerization, in which the coating is deposited on the surface of the substrate via direct monomer polymerization, as described in Wu et al. “Non-Fouling Surfaces Produced by Gas Phase Pulsed Plasma Polymerization of an Ultra Low Molecular With Ethylene Oxide Containing Monomer”, Colloids and Surface, B.-Interfaces, 18, 235 (2000), herein incorporated by reference. In this method, coatings are deposited on solid substrates via plasma polymerization of selected monomers under controlled conditions. The plasma is driven by RF radiation using coaxial external RF electrodes located around the exterior of a cylindrical reactor. Substrates to be coated are preferably located in the reactor between the RF electrodes; however, substrates can be located either before or after the electrodes. The reactor is evacuated to background pressure using a rotary vacuum pump. A fine metering valve is opened to permit vapor of the monomer (or monomer mixtures) to enter the reactor. The pressure and flow rate of the monomer through the reactor is controlled by adjustments of the metering valve and a butterfly control valve (connected to a pressure controller) located downstream of the reactor. In general, the monomer reactor pressures employed range from approximately 50 to 200 mili-torr, although values outside this range can also be utilized. Compounds should have sufficiently high vapor pressures so that the compounds do not have to be heated above room temperature (from about 20 to about 25° C.) to vaporize the compounds. Although the electrodes are located exterior to the reactor, the process of the invention works equally well for electrodes located inside the reactor (i.e. a capacitively coupled system).

The chemical composition of a film obtained during plasma deposition is a strong function of the plasma variables employed, particularly the RF power used to initiate the polymerization processes. It is preferred to operate the plasma process under pulsed conditions, compared to continuous wave (“CW”) operation, because it is possible to employ reasonably large peak powers during the plasma on initiation step while maintaining a low average power over the course of the coating process. Pulsing means that the power to produce the plasma is turned on and off. For example, a plasma deposition carried out at a RF duty cycle of 10 msec on and 200 msec off and a peak power of 25 watts corresponds to an average power of 1.2 watts. The Peak Power may be between about 10 and about 300 watts.

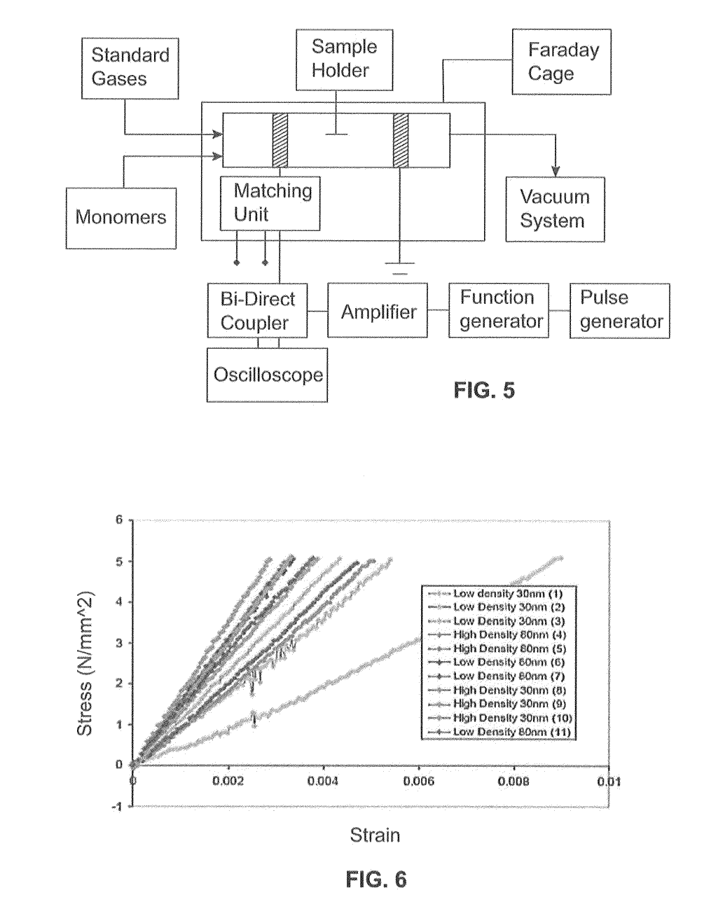

The pulse plasma discharge, based on molecular surface tailoring processing is carried out using 13.6 MHz Radiofrequency (“RF”) power input to create the plasma discharge. Plasma polymerization uses plasma sources to generate a gas discharge that provides energy to activate or fragment monomers, often containing a vinyl group, in order to initiate polymerization. A schematic diagram indicating key aspects of these plasma systems is shown in FIG. 5. A wide variety of monomers are available for use of the plasma source. Based on appropriate choice of monomer and plasma duty cycle employed, conformal films are synthesized with hydrophobic or hydrophilic properties, including functionalized coatings. Coated nanopores using diethylene glycol monovinyl ether (C₆H₁₂O₃) monomer produces hydrogel-like polymer films that are resistant to both protein adsorption and blood platelet adhesion. Other compounds to produce the hydrogel-like polymer films include di(ethylene glycol) divinyl ether, di(ethylene glycol) methyl vinyl ether, di(ethylene glycol) ethyl ether acrylate, and trimethylolpropane diallyl ether. The most preferred compound is di(ethylene glycol) vinyl ether. However, other monomers, including functional monomers such as allyl alcohol, permitting drug attachment, including heparin, can be adapted to the process, allowing identification of the surface composition, which is most preferable with respect to both non-fouling and prevention of serum leakage in the exchanger. Biologically non-fouling means that proteins, lipids and cells will not adhere to the surface of a device.

The plasma films are characterized using spectroscopic and other measurements. These include XPS and FT-IR spectroscopy along with microscopic analyses using AFM, SEM, and HRTEM. Surface wettability is determined using RAM-Hart sessle drop goniometry. Film thickness and refractive index is determined using a laser profilometer and ellipsometer, respectively. Gaseous diffusion through the membrane, before and after plasma modification, is determined using systems and procedures as described in Ley et al. “Permeation rates of low molecular weight gases through plasma modified membranes” J. Of Membrane Science 226, 213-226, (2004), herein incorporated by reference.

Pulsed plasma polymerization process regulates gas permeation rates through nanoporous membrane 20 by the polymer films on the nanopores 22. The permeation rates were shown to be functions of both the composition and thickness of the polymer films deposited on the membranes during the plasma initiated deposition processes. The polymer films preventing liquid penetration through the pores while simultaneously discouraging deposition of matter (ie. bio-fouling) in the nanopores 22. Slow water adsorption may occur on a hydrophobic fluorocarbon surface of the nanoporous membrane when that surface has an underlayer of a hydrophilic polymer, such as poly-N vinyl pyrrolidone, and the nanopore internal architecture has ridges that would enhance water penetration. Water penetration may be eliminated by removing the underlayer of hydrophilic polymers and removing ridges on the nanopores. Biomolecule adsorption/denaturation and platelet adhesion/activation is eliminated, which would otherwise impede gas flow and initiate thrombus formation.

In one embodiment of the invention, a super hydrophobic film is generated via pulsed plasma polymerization of perfluorinated monomers. The surfaces of the perfluorinated monomers are non-wettable with sessile drop water contact angles in excess of 170⁰. Additionally, the perfluorinated monomers surfaces include zero hysteresis in advancing/receding contact angle studies, which rejects water at/in the nanoporous channels 10 for the long term. Super hydrophobic films deposited on the SiN nanoporous membranes will be evaluated. The film thickness and film cross-link density will be sufficient to render the nanoporous membranes impermeable to water while simultaneously permitting adequate flow of the non-polar O₂ and CO₂ molecules. The initial evaluations will involve monitoring the contact angle of a water droplet on the surface of the perfluorinated film as a function of time. Subsequently, the coated nanoporous membranes will be subjected to an increasing hydrostatic pressure as applied by increasing the height of water in a column above the membrane, which is a standard procedure used industrially to measure the wettability of materials. The perfluorinated film prevents water penetration at hydrostatic pressures that significantly exceed the pressures present under blood flow conditions.

In another embodiment of the invention, deposition of a polyethylene glycol (“PEG”) film on top of the super hydrophobic film on the blood contacting side of the nanoporous membrane. PEG minimizes and eliminates biological fouling of the nanoporous membrane on the blood contacting surface, i.e. along the nanoporous membrane 20 in contact with the blood channel 40. PEG films are effective in sharply reducing biomolecule adsorption on surfaces, such as pulse plasma depositing diethylene glycol vinyl ether monomers. The pulsed plasma polymerization will maximize the retention of the ether content of the monomer, and the non-fouling property of the polymer films deposited on the blood contacting side. This permits adjustment of the film compositions and thicknesses with respect to optimizing non-fouling without compromising gas permeation rates. If water does penetrate the PEG layer, the water will be arrested at the super hydrophobic interface. The efficacy of this approach will be evaluated initially using a variety of biomolecule-containing solutions (e.g. proteins, peptides, sugars, etc.) and more complex mixtures, including some containing red blood cells, will be used to examine possible platelet depositions. The extent of non-fouling will be assessed using radio- or fluorescence labeled molecules. Functionalization of the polymer-coated exchanger blood channels is also feasible, for example, with an allyl alcohol coating. This enables the attachment of biomolecules favorable to influencing the biocompatibility of the exchanger, such as heparin, by various schemes well known in the art. In addition, treatments can be done with other small molecule drugs, such as those inhibiting the inflammatory response, e.g., paclitaxel, curcumin, everolimus, etc.

In one embodiment of the invention, the nanoporous membrane exchanger 100 can be coupled to a miniaturized chandler loop system, employing flowing surrogate fluids and fresh whole blood, for evaluation of oxygen and carbon dioxide exchange efficiency. In another embodiment of the invention, a system for evaluation of the influence of blood proteins, platelets, leukocytes, and red cells on fouling of the exchange surfaces is contemplated.

### Membrane Mechanical Strength

The mechanical integrity of the nanoporous membrane 20 and the integrated exchanger device under operational conditions is maintained. The nanoporous membrane 20 mechanical strength is characterized for both polycarbonate track-etched membrane and silicon nitride membranes. Stress-strain tests of nano-pored polycarbonate track-etched membranes using an Instron 5565 device (Grove City, Pa.). From stress-strain response curves, the membrane stiffness (i.e. the Young's modulus) and failure strength, failure strain levels are determined. A load cell of 50 Newtons was used with a load rate set at 1 mm/min. Using a template, the membrane is cut into a “dog-bone” shape with dimension of 15 mm×38 mm (width×length), with the thickness approximately 6 μm. After mounting the specimen to the pneumatic-controlled grips, the load at a rate of 1 mm/min is increased until the membrane specimen broke. Load-deflection data were converted into stress-strain curves.

Stress-strain curves of all coated and uncoated membranes were calculated. Closer examination of the toe region of the graph, as shown in FIG. 6, shows that when holding coating thickness constant at 30 nm, high crosslink density coating leads to higher Young's modulus. However, at 80 nm thickness, a significant difference in Young's modulus due to crosslink density differences is not shown. For both high and low crosslink density, there were no significant differences in Young's modulus between 30 and 80 nm thickness groups.

### Strength of Silicon Nitride Membrane

The silicon nitride membranes 28 were tested for their strength using a pressure sensor characterization setup. A load cell can load any particular area up to 10 grams in weight, with a resolution in nanograms. The sample was placed in the stage and the probe was moved down on to the sample in steps of 2 μm up to 50 μm. The diameter of the probe is a known value. Using the diameter of the probe, the pressure exerted on the membrane was calculated. The non-porous membranes were subjected to pressures ranging from 500 Pascal to 2.12×10⁵ Pascal. There was no visible deformation or damage caused on the membrane, which shows that the membrane can be subjected to high pressures without any appreciable damage to the membrane.

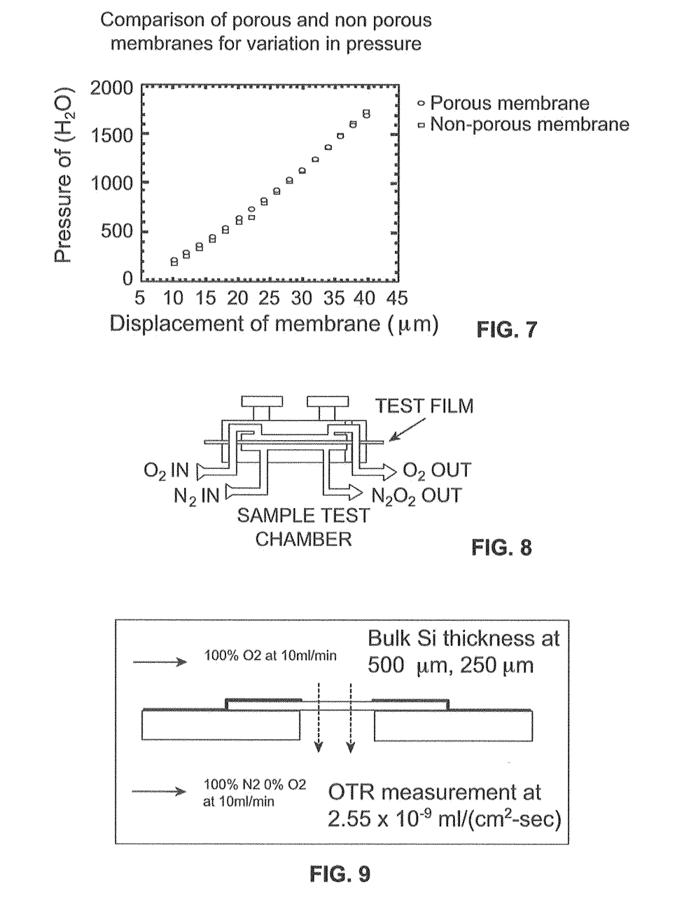

The membrane 28 was drilled with nanopores 22 of four different diameters to form the nanoporous membrane 20. The diameters of the nanopores 22 were approximately 4, 8, 12 and 13 microns. A total of 205 nanopores were drilled in the membrane to make the nanoporous membrane 20 approximately 0.61% porous. The nanoporous membrane 20 was subjected to the mechanical strength test as the non-porous membrane. The nanoporous membrane 20 was subjected to pressures ranging from 574 Pascal to 2.41×10⁵ Pascal. The variation of pressure with respect to the displacement of the nanoporous membrane 20 compared to the variation of pressure with respect to the nonporous membrane 28 displacement nonporous membrane 28 is shown in FIG. 7. There is no appreciable change in mechanical strength with a low porosity. The results are verified on the indentation test simulated using ANSYS® Finite Element Analysis (“FEM”).

Using the SolidWorks CAD modeling system, a solid model consists of the indenter and the silicon nitride membrane. The dimension of the membrane is 1,100×1,100×1.4 μm whereas the indenter has a diameter of 500 μm. The cylindrical part of the indenter is excluded from the model to simplify without introducing errors. With a hemispherical-shaped head, the indenter is perpendicular to the membrane upper surface. The solid model was imported into ANSYS where a finite element model is constructed. All four thickness edges of the membrane were constrained from any movement. A pressure load of 0.166 Newton/mm² was applied to the upper surface of the indenter. By requiring the Young's modulus for the indenter to be 10 times that of the membrane, the experimental data of pressure load and membrane predicts the Young's modulus for the silicon nitride membrane. The Young's modulus for the silicon nitride membrane has been reported to be 0.38×10⁶ Newton/mm². The Young's modulus for the silicon nitride membrane is 0.304×10⁷ Newton/mm². The idealized frictionless contact could contribute to the overestimation in Young's modulus.

### Membrane Permeability

The nanoporous membranes 20 separates the blood channels 40 and the gas channels 30, where gas exchange takes place across the nanoporous membrane 20. The properties of nanoporous membranes ensure adequate strength, gas permeability, resistance to water penetration and biocompatibility. Further, characterization of different types of biocompatible polymer coatings and their respective thicknesses affect the membrane permeability to O₂ and CO₂, which provide the capability to modulate gas exchange. The permeability of nano-pored polycarbonate track-etched (“PCTE”) membranes characterizes the effects of pore-diameter, polymer coating types, crosslink density, coating thickness, as well as permeant gas. Contact angles measurements from polymer coatings are compared to assess the degrees of hydrophobicity and to ensure the membrane resistance to “wet-out” is adequate.

### Permeability of a Nanoporous Polycarbonate Track-Etched Membrane

Polycarbonate track-etched membranes (“PCTE”) of 50 nm and 100 nm nanopore size, were surface treated with either Vinyl Acetic acid (“VAA”) or Perfluorohexane (“C₆F₁₄”) using a variable duty cycle pulsed plasma polymerization technique. The surface treatment affects the gas permeation properties of the PCTE, which is similarly applied to the silicon nitride nanoporous membrane 20. Controllably varied plasma coating thickness resulted in gradual reduction of O₂ and CO₂ permeability, as thickness increased from 10 nm to 100 nm. Plasma coating material, permeant gas, membrane nanopore size, and crosslink density can be varied to modulate the permeation properties of the PCTE. The results show a wide range of permeabilities are achievable via this method. O₂ was more permeable than CO₂. Varying the crosslink density had a noticeable effect on the surface wettability as well as the gas permeability. The results from advancing/receding contact angle measurements indicate a much more hydrophobic character when the surface was coated with C₆F₁₄ compared to the uncoated and VAA coated samples.

Both experiment and calculation show that the nano-pored silicon nitride membrane oxygenates blood. The modified PCTE membranes have sufficient O₂ and CO₂ transfer blood oxygenation. The plasma polymerization process can modulate the gas permeability characteristics of the PCTE membranes and also alter the membrane surface to improve performance and blood oxygenation.

The PCTE membrane included a 47 mm diameter disk, with either 50 nm or 100 nm nanopore sizes, and a thickness of 6 μm±0.6 μm. The PCTE membranes were subsequently coated either with C₆F₁₄ or Vinyl Acetic Acid (CH₂═CHCH₂COOH is abbreviated as “VAA”) via the pulsed plasma polymerization technique. A gas permeability apparatus was built to measure and compare the O₂ and CO₂ permeabilities of the PCTE membranes coated to varying conditions (thicknesses, crosslink density). The flowrate vs. pressure curves were obtained to calculate the membrane permeability. Surface hydrophobicity characteristics of the PCTE membranes using the advancing/receding contact angle technique are examined. Sample specimens are scanned using a scanning electron microscope (“SEM”) to examine the effects of coatings on nanopore size and nanopore structures.

PCTE membranes were coated under varying conditions using variable duty cycle pulsed plasma polymerization technique. The sample is placed in a plasma reactor and exposed to a partially ionized gas plasma produced by a high frequency electric field (on ˜10 msec/off ˜90 msec). Reactive species produced during the plasma on times continue to react with undissociated monomer during the plasma off times, resulting in deposition of thin polymer films on the membrane surface. The polymer films so formed provide a conformal, pin hole free coating. Pores orthogonal to the membrane surface can be partially coated. Coating thickness can be adjusted via plasma excitation conditions. When the coating is applied to coat the nanopore walls, the pore size can be controllably reduced, such that the gas permeability can also be controllably reduced. Gas flow rates as a function of applied pressure through coated and uncoated membranes were studied with a simple gas permeation apparatus. The advancing/receding contact angle measurements were taken to compare nanoporous surfaces with hydrophilic and hydrophobic coatings. PCTE membranes containing 50 nm or 100 nm nanopore sizes were plasma coated with varying thicknesses from 10 nm to 100 nm with either Vinyl Acetic Acid (VAA) or Perfluorohexane (C₆F₁₄). Coated membranes were placed in vacuum for 2-3 days in order to remove any unreacted monomer content and subsequently set aside for gas permeation experiments.

### Gas Permeability

The gas permeability apparatus uses ¼″and ⅛″ steel Swagelok tubing as well as ⅛″ flexible tubing that connects a gas cylinder source of either Oxygen gas or Carbon Dioxide gas (Airgas Southwest, Arlington, Tex.) to a digital pressure gauge (Cole Parmer, Ill.). From the gauge, the tubing feeds into a correlated flowmeter (Cole Parmer, Ill.) and immediately into the membrane chamber in which the membrane under study is securely sealed and mounted. A porous metal disc inside the membrane chamber is used to support the PCTE membranes but does not have any noticeable impedance to gas flowrate. From the membrane chamber, the tubing connects to a glass bubble flowmeter (Bubble-O-Meter, Ohio). The permeant gas exits the regulator, flows into the membrane chamber with the mounted membrane, through the membrane, and finally into the bubble flowmeter. A soap bubble is introduced into the gas stream to calculate the flowrate by timing the rise of the soap bubble through a known volume increment.

Membranes are placed and sealed into the membrane holder, and then oxygen is passed through for about two minutes to remove any residual gases. Although the diameter of membranes is 47 mm the effective diameter in the flow path once mounted was only 36 mm. Next, the vent was closed and a pressure of 0.25 psi was applied to the membrane. The resulting flowrate was measured. Five flowrate measurements were taken at a given applied pressure. The pressure was then incrementally adjusted from 0.25 psi up to approximately 3.5 psi to obtain accurate measurements. The membrane was then either removed from the membrane holder or a different gas was tested. Either Oxygen (O₂) or Carbon Dioxide (CO₂) was used as permeant gases for these studies. Tested membranes were examined by SEM or contact angle measurement.

The flowrate of the gas exiting the membrane was experimentally measured. In order to determine the gas permeability from the flowrate vs. pressure curve, the slope of the linear trendline was calculated and the following equation was used:

$$J = KA \frac{Δ P}{L} \qquad ( 1 )$$

Where J=flowrate (mL/s); A=membrane area exposed to gas stream (cm²) ΔP=pressure gradient (cmHg) L=thickness of membrane (cm) K=Permeability (cm³*cm*cm⁻²*s⁻¹*cmHg⁻¹)

Permeability is expressed in Barrers where 1 Barrer=10⁻¹⁰ cm³*cm*cm⁻²*s⁻¹*cmHg⁻¹. Since, in the preceding equation the volume flowrate through the membrane is proportional to the pressure difference applied across the membrane, the permeability may be obtained from the slope of the flowrate vs. pressure line for a given membrane sample. For a plot of J vs. ΔP, the slope is equal to:

$$Slope = \frac{KA}{L} \qquad ( 2 )$$

So the permeability, K is:

$$K = Slope * \frac{L}{A} \qquad ( 2.1 )$$

A Rame-Hart Goniometer (Rame-Hart Instrument Co., Netcong, N.J.) measures the water contact angle on uncoated and pulsed plasma coated PCTE membranes. Advancing/receding contact angle measurements were taken. The featured membrane was taped onto a clean glass slide so that the membrane lay extremely flat. For advancing/receding measurements, a 2 μL water droplet was placed on the membrane surface. With the pipette tip submerged into the droplet, increments of 2 μL were released into the droplet causing an increasingly larger water droplet. The contact angle was recorded at each volume increment. For the receding angle, the reverse process was performed: the micropipette was used to withdraw 2 μL increments of water back from the droplet until the droplet was gone or the contact angle dropped below 200. The contact angle again was recorded at each volume increment. The resulting advancing/receding contact angle plots were used to compare hydrophobicity of membrane surfaces.

SEM visualized the mircoscale structure of the nanoporous PCTE plasma coated membranes with various coating thickness. The membranes were first gold sputter-coated with a thickness approximately 70 angstroms, using a MRC sputter coater system (Semicore, Calif.) to prevent charging. The membranes were then mounted onto sample studs and placed in the Zeiss Supra VP Scanning Electron Microscope (Zeiss, N.Y.). Images were taken at 5 kV and 35 kx and 50 k magnification.

For the PCTE membrane with a diameter of 47 mm and an effective diameter of 36 mm, and a thickness of 6 μm+/−0.6 μm, a coating thickness of 10-60 nm for 50 nm nanopore sized membranes resulted in gas permeation, contact angle, and SEM visualization. When the PCTE membrane was coated with VAA, there was gas permeation, contact angle, and SEM visualization. When the coating material was C₆F₁₄, there was gas permeation, contact angle, and water contact visualization. Both oxygen and carbon dioxide were permeant gases with gas permeation. The nanopore diameter of 50 nm and 100 nm maintained gas permeation and contact angle. The low, medium, and high crosslink density maintained gas permeation and contact angle.

The coating thickness ranges from approximately 10-100 nm, where the thicker the coating the larger the nanopore size reduction, and thus the lower the permeability of the membrane. The polymer films produced from C₆F₁₄ are much more hydrophobic than VAA, and thus more effectively able to prevent water penetration into pores. And the uncoated nanopore actually contains hydrophilic wetting agent. Between the permeant gases O₂ and CO₂, O₂ is more permeable than CO₂. And between the original uncoated membranes nanopore sizes of 50 nm and 100 nm, 100 nm nanopore-sized membranes are much more permeable than the 50 nm pore sized membranes. The crosslink density of the plasma deposited polymer films include 3 levels, low, medium, and high, where the more highly crosslinked caused the lower permeability, and the more highly crosslinked caused an increased hydrophobicity. Therefore, oxygen permeability is reduced as crosslink density of the coating is increased. Such PCTE parameters for coatings and nanopore size can be used on the silicon nitride membrane.

The gas permeation rates through the membranes are modulated via deposition of polymer films, whose thickness and cross-link density can be controlled to regulate gas flow rates. Alternative volatile monomers, in addition to VAA or C₆F₁₄, can be employed to modulate the nanopore size, and thus permeation rates, via the pulsed plasma deposition process. In one embodiment of the invention, deposition of the polymer film on the nanoporous membranes is by a variable duty cycle pulsed plasma deposition process.

### Permeability Measurement of Silicon

An Oxygen Permeation Analyzer (OTR 8001, Illinois Instruments) measures oxygen permeation through the membranes. The analyzer measures the oxygen transmission rate across a membrane based on the concentration difference. A schematic diagram of the test chamber is shown in FIG. 8. The test membrane film is mounted to a window that separates two gas channels. Oxygen (O₂) at 100% flows through the upper channel; whereas nitrogen (N₂) at 100% flows through the lower channel. Gas flows are regulated such that two channels have the same flow rates with zero convective pressure across the membrane. Oxygen molecules diffuse through the membrane due to concentration differences. Oxygen Transmission Rate (“OTR”) is a permeability measure for the amount of oxygen that diffuses across the membrane per unit time, per unit area.

For the measurement, a steel plate is mounted to the ¼ wafer (˜250 or 500 μm). A masking foil (one side self-adhesive) is applied to seal the surface of the ¼ wafer and the steel plate except the circular region (area: 5 cm²). A sensor at the lower channel detects the amount of oxygen molecules and registers it to a connected PC at a specified sampling rate (every 5 minutes). Extra caution is needed when applying masking foil to eliminate any possible gas leak due to trapped air pockets. Grease was also applied to the outer edge of the wafer in an effort to eliminate any air pockets. A cross-section of the membrane, steel plate, and masking foil is shown in FIG. 9.

Bulk silicon with no nanopores included a value of ˜2.55×10⁻⁹ ml/(cm²-sec). The measurement is extremely sensitive to any gas leak, where a small amount of gas leak causes large errors to “the true permeability” measured. The permeability of the PCTE porous membrane with a thickness of 6 μm is 1×10⁻¹ m/(cm²-sec-cmHg) and commercial hollow fiber polypropylene membranes has a permeability of 2˜9×10⁻¹ (cm²-sec-cmHg). The measurements of bulk silicon showed almost no gas permeation compared to PCTE and commercial hollow fiber polypropylene membranes.

The permeability of silicon nitride membranes that have uncoated nanopores and the permeability of coated silicon nitride membranes can be characterized in a similar fashion. These characterizations can determine the optimum coating thickness for the nanopores, as well as to provide optimum gas exchange efficiency.

### EXAMPLES

The following examples are put forth so as to provide those of ordinary skill in the art with a complete disclosure and description of how the compositions, compositions, articles, devices, systems, and/or methods claimed herein are made and evaluated, and are intended to be purely exemplary and are not intended to limit the scope of compositions, compositions, articles, devices, systems, and/or methods. Efforts have been made to ensure accuracy with respect to numbers (e.g., amounts, temperature, etc.), but some errors and deviations should be accounted for.

### Nanoporous Channel Designs

The nanoporous membrane exchanger 100 includes several nanoporous channel designs, which include, but are not limited to, a dome channel design 200, a roof-top dome channel design 300, a roof-top channel design 400, and a roof-top channel design 500.

### Dome Channel Design

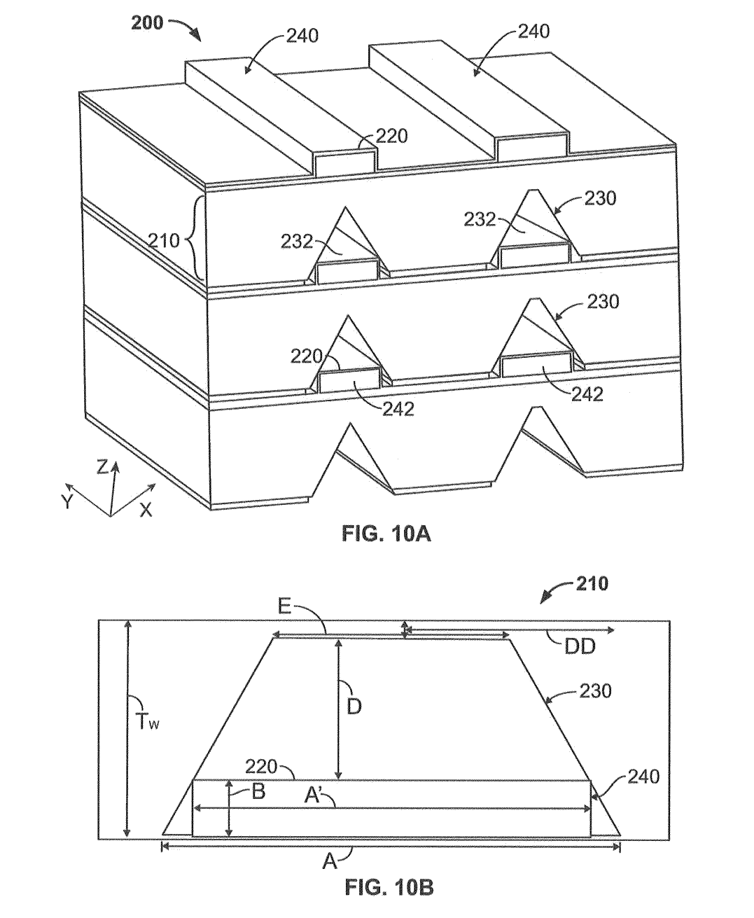

The dome channel design 200 is shown in FIG. 10A, which comprises a plurality of dome channels 210. The dome channel 210 includes a nanoporous membrane 220, a gas-channel 230, and a blood channel 240. The gas 232 is conducted through the gas-channel 230 and the blood 242 is conducted through the blood channel 240 as to permit oxygenation of blood through the nanoporous membrane 220.

The height B of the blood channel 240 and the height D of the gas channel 230 can be varied to obtain a balance between the blood and the gas volumes. The height of the blood channel B may be 5, 8, or 10 μm in order to permit the red blood cell deformation of torpedo-to-parachute shape to substantially increase oxygenation efficiency. The height of the gas channel is varied keeping the height of the blood channel a constant to obtain an optimum balance in the blood and gas volumes. The dome channel 210 includes bulk micromachining along the silicon crystallographic <100> direction in conjunction with surface micromachining under a thin sputter-deposited polycrystalline silicon layer, as described previously for the formation of the nanoporous channel 10. A layer doped with boron atoms act as an etch-stop to define the base of the blood channels 240 made by the surface micromachining. Multiple dome channels 210 similarly processed are bonded and stacked up to build the exchanger. Biocompatible bonding materials such as PPMA and PEBMA have an adhesive strength for this purpose.

The width at the top of the gas channel E is the factor which determines the volume of the gas channel. In one embodiment of the invention, E is varied from 0-25 μm in steps of 0.1 μm. The corresponding values of the other dependant parameters were calculated. The values of the parameters were chosen such that the ratio of blood volume to gas volume is balanced. A balanced ratio of blood volume to gas volume is obtained with the height of the gas channel at 10 μm. The ratio of blood volume to gas volume ranges from 90.91% to 79.86%, when the value of the parameter E is varied from 0 μm-5 μm with the gas channel at 10 μm. Varying the parameter E (i.e.) the width of the gas channel from 0 μm-5 μm, the balanced volumes of blood and gas is obtained. The ratio of the surface area of interaction to blood volume is 0.1 μm⁻¹ in one embodiment of the invention.

The cross section of the dome channel 210 is show in FIG. 10B. The region which is shaded in red is the blood channel 240 and the region shaded in green is the gas channel 230. T_{w} is the thickness of the wafer. T_{w} is at a fixed thickness and may be 40 μm in one embodiment of the invention. A is the width at the bottom, which may be dependent upon the blood-gas volume ratio. A′ is the width of the blood channel and dependent upon the blood-gas volume ratio desired. E is the width of the gas channel 230 at the top, which is independent of the blood-gas volume ratio. E may be 0 to 25 μm in increments of 0.1 μm. D is the height of the gas channel 230, which is dependent upon the blood-gas volume ratio. B is the height of the blood channel 240, which is independent of the blood-gas volume ratio. B may be 5 μm, 8 μm, 10 μm. DD is the diffusion depth, which is independent of the blood-gas volume ratio. DD may be 8 μm in one embodiment of the invention.

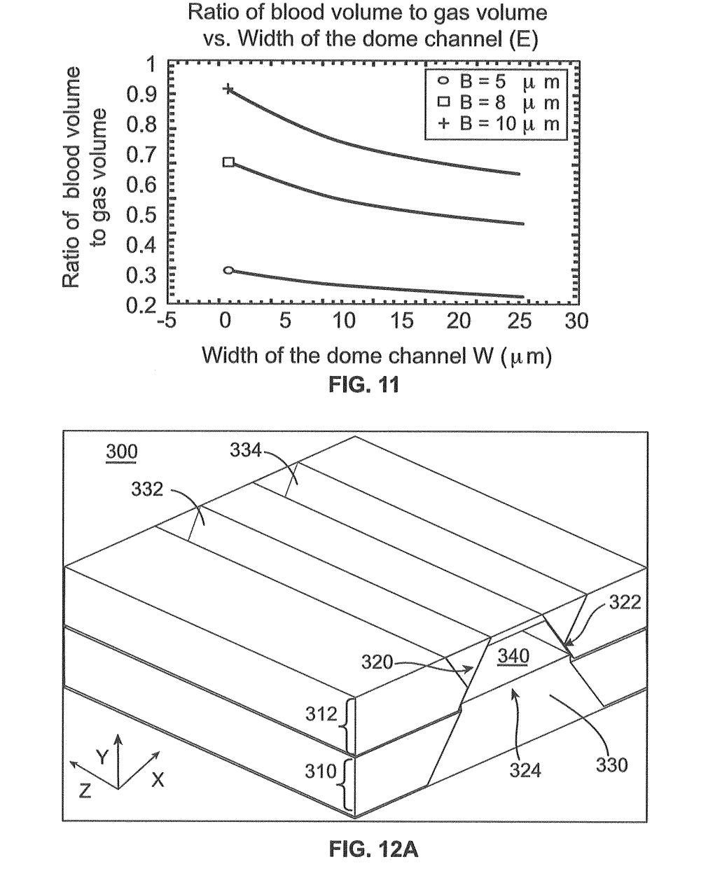

FIG. 11 shows the variation of blood to gas volume ratio to the width of the gas channel (E) for 3 different height of blood channel. The blood to gas volume ratio varies from 90% to 62%. The blood to gas volume ratio increases with the blood channel height B. The gas exchange surface area to blood volume ratio is 0.1 μm⁻¹.

### Roof-Top/Dome Channel Design

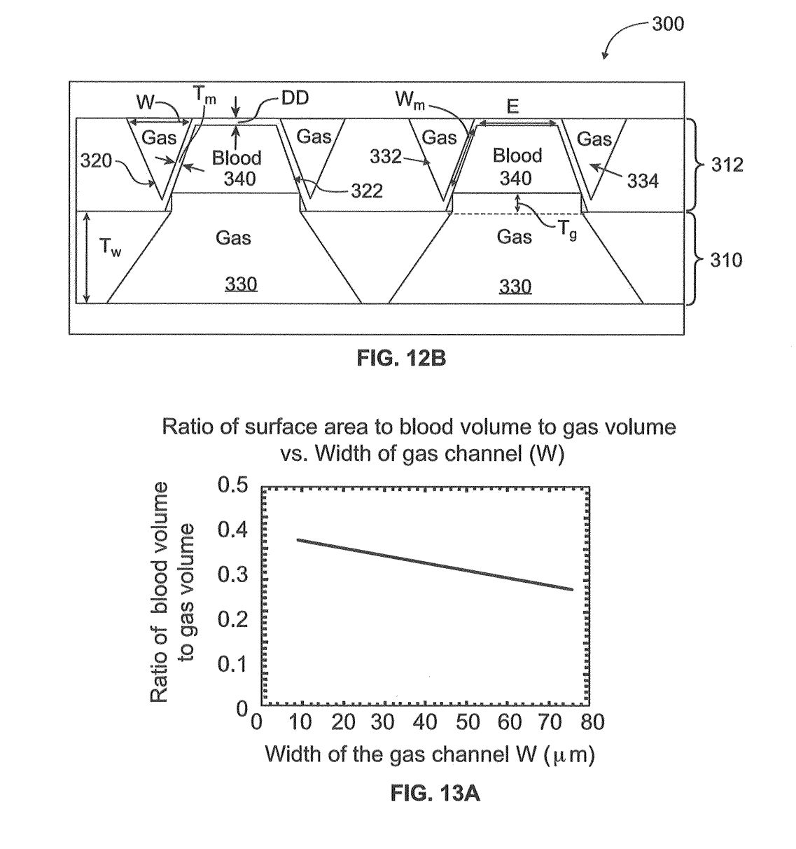

As shown in FIG. 12A, the roof-top/dome channel design 300 includes at least one dome channel 310 connected to at least one roof top channel 312 to form at least one blood channel 340 and at least three gas channels 330, 332, 334. The roof-top/dome channel design 300 includes two different wafers to be processed. The dome channel 310 is similarly processed to the to the dome channel 210, except for the lack of the etch-stop layer. The roof-top channel 312 is produced using an anisotropic wet etchant, that preferentially etches (100) crystallographic planes but not (111) planes in the silicon layer 50, thus leaving a first and a second nanoporous membrane layer 320 and 322 at a 54.7 angle, and a poly-silicon layer 324. Bonding the dome channel 310 and the roof-top channel 312 achieves the blood and gas channels and this technique takes advantage of the inherent mechanical strength of the silicon crystal.

The cross section of the roof top/dome channel design 300 is shown in the FIG. 12B. The region which is shaded in red is the blood channel 340 and the region shaded in white are the gas channels 330, 332, and 334. T_{w} is the thickness of the silicon layer, which is fixed at 40 μm in one embodiment of the invention. W is the width of the gas channels 332 and 334 at the top of the roof-top channel 312. W may be independent from the blood-gas volume ratio and varied from 10 μm-75 μm. Tₘ is the thickness of the nanoporous membranes 320 and 322, which is fixed at 0.8 μm in one embodiment of the invention. T_{g} is the height of the gas channel 330 in the dome channel 310, which is independent of the blood-gas volume ratio at 5 μm. Wₘ is the width of the nanoporous membranes 320 and 322, which is dependent upon the blood-gas volume ratio. DD is the diffusion depth at the top of the blood channel 340, which is independent of the blood-gas volume ratio at 5 μm. E is the width at top of blood channel 340, which is independent of the blood-gas volume ratio at 50 μm.

The height of the blood channel 340 and the width of two of the gas channels 330 and 332 can be varied to obtain a balance between the blood and the gas volumes. The height of the third gas channel depends on the height of the second wafer. The width of the gas channel (W) was varied form 10 μm-75 μm for calculating the design parameters. The width of the gas channel was varied keeping the height of the blood channel a constant to obtain an optimum balance in the blood and gas volumes.

The width W of the gas channel at top is the parameter that determines the volume of the gas channels 332 and 334 in the roof-top channel 312. W was varied from 10 μm-75 μm in steps of 0.5 μm. The corresponding values of the other dependant parameters were calculated. The values of the parameters were chosen such that the ratio of blood volume to gas volume is balanced. The balanced ratio of blood volume to gas volume is obtained when the width W of the gas channel is 32 μm. The ratio of blood volume to gas volume is approximately 33%, when the height of the blood channel is maintained at 30 μm. Thus, having the parameter W, i.e., the width of the gas channel, approximately varying in the range of 32 μm, the balanced volumes of blood and gas is obtained. The ratio of the surface area of interaction to blood volume varies in this design with the width of the gas channel. When the width W of the gas channels 332 and 334 was varied from 10 μm-75 μm, the surface area of interaction to blood volume varies from 0.096-0.139 μm⁻¹. When the width of the gas channels 332 and 334 is 32 μm, it results in a balanced ratio of blood volume to gas volume, and the surface area of interaction is 0.1108 μm⁻¹.

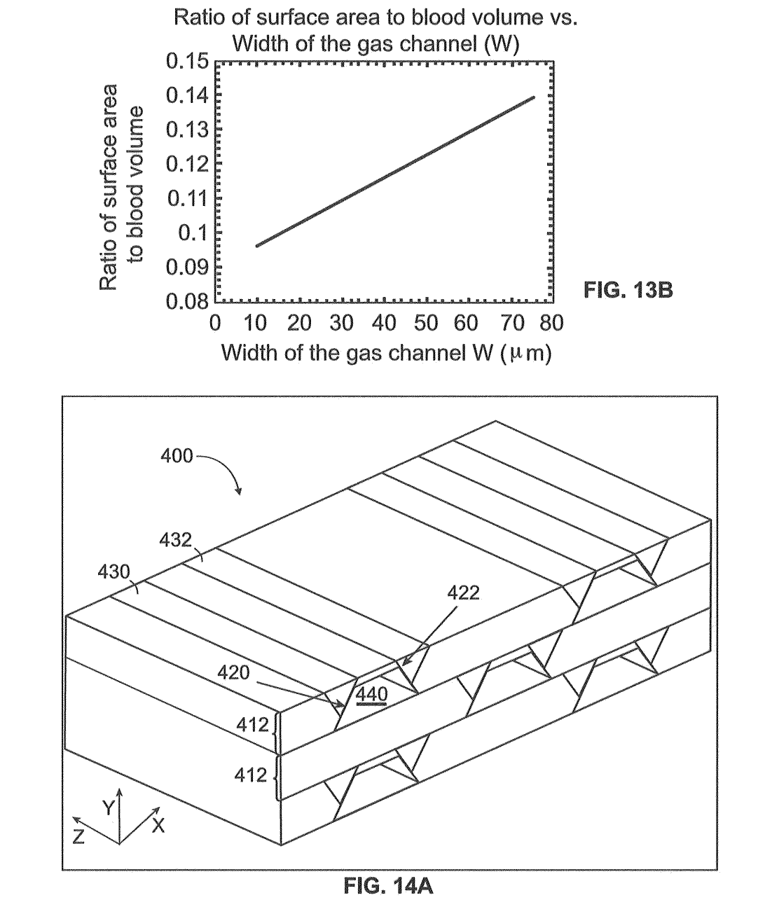

FIG. 13A shows the variation of blood to gas volume ratio with the width W of the gas channels 332 and 334. FIG. 13B gives the surface area of interaction to the blood volume as the gas channel W is changed.

### Roof-Top Channel Design

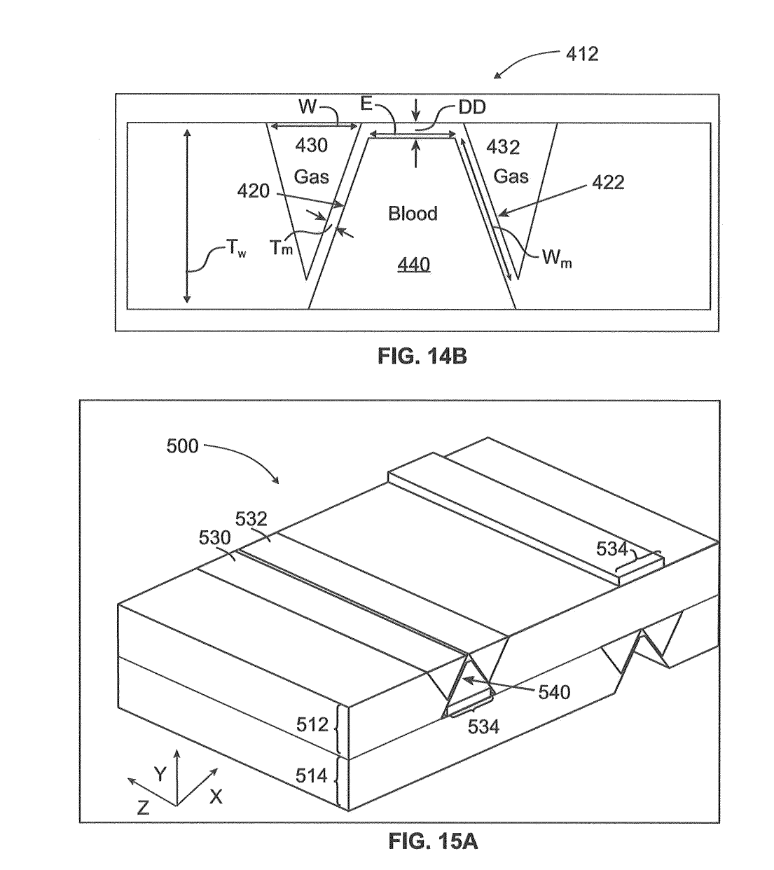

As shown in FIG. 14A, the roof-top channel design 400 comprises at least two roof top channels 412. The roof top channels 412 include at least one blood channel 440 and at least two gas channels 430 and 432, where at least two nanoporous membranes 420 and 422 are between the blood channel 440 and the gas channels 430 and 432. The alignment of the roof top channels 412 is staggered such that the bottom of the blood channel 440 is bonded and sealed by the roof top channel 412 below the bottom of the blood channel 440. The roof top channel 412 is produced in a similar manner of the roof top channel 312 described previously.

A cross-section of the roof top channel 412 is shown in FIG. 14B. T_{w} is the thickness of the roof top channel 412, which is fixed with respect to the blood-gas volume ratio at 40 μm in one embodiment of the invention. W is the width at the top of the gas channels 430 and 432, which is independent of the blood gas ratio and varied from 10-56.5 μm in steps of 0.5 μm. Tₘ is the thickness of the nanoporous membranes 420 and 422, which is fixed with respect to the blood-gas volume ratio at 0.8 μm in one embodiment of the invention. Wₘ is the width of the nanoporous membranes 420 and 422, which is dependent upon the blood-gas volume ratio. DD is the diffusion depth, which is independent of the blood-gas volume ratio at 7 μm. E is the width at top of blood channel 440, which is independent of the blood-gas volume ratio at 50 μm. The height of the blood channel 440 and the width W of two of the gas channels 430 and 432 can be varied to obtain a balance between the blood and the gas volumes. The width W of the gas channels 430 and 432 was varied by keeping the height of the blood channel a constant to obtain an optimum balance in the blood and gas volumes.

The width W at the top of the gas channels 430 and 432 is the parameter that determines the volume of the gas channel and was varied from 10 μm-56.5 μm in steps of 0.5 μm. The corresponding values of the other dependant parameters were calculated and the values of the parameters were chosen such that the ratio of blood volume to gas volume is balanced. A balanced ratio of blood volume to gas volume is obtained when the width of the gas channel is 50 μm. The ratio of blood volume to gas volume is approximately 137%, when the value of the parameter height of the blood channel is maintained at 33 μm. Varying the parameter W, i.e. the width of the gas channel, from 50 μm, the balanced volumes of blood and gas is obtained. The surface area of interaction of blood with gas volume varies in this design with varying values of the width if the gas channel. When the width of the gas channel is varied from 10 μm-75 μm the value of the surface area of interaction varies from 0.0065 to 0.033 μm⁻¹. When the width of the gas channel is 50 μm, a balanced ratio of blood volume to gas volume the surface area of interaction is 0.0299 μm⁻¹ is obtained. The region which is shaded in red is the blood channel and the region shaded in green is the gas channel.

### Roof-Top Channel Design 500

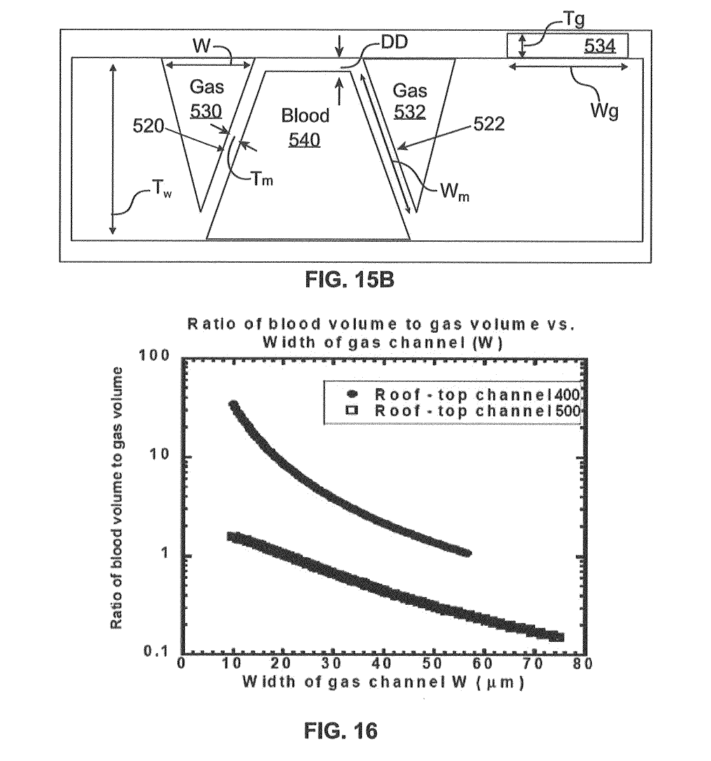

Another embodiment of the roof-top channel design 500 is shown in FIG. 15A. The roof top channel design comprises at least two roof top channels 512 and 514. The two roof top channels 512 and 514 include at least one blood channel 540, at least three gas channels 530, 532, and 534, and at least two nanoporous membranes 520 and 522. The nanoporous membranes 520 and 522 are located between the gas channels 530, 532 and the blood channel 540. The gas channel 534 on the second roof top channel 514 is aligned on the bottom of the blood channel 540 in the first roof top channel 512, such that the blood channel 540 is sealed by the gas channel 534. The alignment and the bonding of the at least two roof top channels 512 produces roof top channel design 500.

FIG. 15B shows a cross section of the roof top channels 512 and 514. The region which is shaded in red is the blood channel 540 and the regions shaded in white are the gas channels 530, 532, and 534. T_{w} is the thickness of the roof top channels 512 and 514, which is fixed at 40 μm. W is the width of the top of the gas channels 530 and 532, which is independent of the blood gas ratio and varied from 10-75 μm in steps of 0.5 μm. Tₘ is the thickness of the nanoporous membranes 520 and 522, which is fixed at 0.8 μm. Wₘ is the width of the nanoporous membranes 520 and 522, which is dependent on the blood gas ratio. DD is the diffusion depth, which is independent of the blood gas ratio at 5 μm. E is the width at top of the blood channel 540, which is independent of the blood gas ratio at 5 μm. T_{g} is the thickness of the gas channel 534, which is independent of the blood gas ratio at 8 μm. W_{g} is the width of the gas channel 534, which is dependent upon the blood gas ratio. The height of the blood channel 540 and the width of two of the gas channels 530 and 532 can be varied to obtain a balance between the blood and the gas volumes. The height of the gas channel 534 can also be varied. The width of the gas channel 534 was varied keeping the height of the blood channel 540 and the height of the gas channel 534 a constant to obtain an optimum balance in the blood and gas volumes.

The width of the gas channel at the top (W) is the parameter that determines the volume of the gas channel and was varied from 10-75 μm in steps of 0.5 μm in one embodiment of the invention. The values of the parameters were chosen such that the ratio of blood volume to gas volume is balanced. In one embodiment of the invention, a balanced ratio of blood volume to gas volume when the width of the gas channels 530 and 532 (W) is 35 μm. The ratio of blood volume to gas volume is approximately 54%, when the height of the blood channel is maintained at 27 μm. Thus, having the parameter W, i.e., the width of the gas channels 530 and 532, varying approximately 35 μm gives the balanced volumes of blood and gas. The surface area of interaction to blood volume ratio varies in the design with the width W of the gas channels 530 and 532. When the width W of the gas channels 530 and 532 varies from 10-75 μm, the surface area of interaction to blood volume ratio is approximately 0.168 μm⁻¹.

A comparison of the roof top channel design 400 and roof top channel design 500 for variation in blood to gas volume ratio as a function of the gas channel width, is shown in FIG. 16.

### Pressure-Blood-Flow Relationship

Blood flow through the blood channels can be described by:

∇p+ μ̅∇²V=0 (3)

where ∇p denotes the driving pressure gradient, μ̅ denotes the effective viscosity of the blood, and V is blood velocity. Equation (3) is solved for velocity distribution using a Galerkin-based finite element model. The effective viscosity μ̅ depends on the local instantaneous shear-rate according to the Casson equation. The resulting velocity is integrated over the blood channel cross-section to obtain the pressure-flow relationship. Blood channel hematocrit (“Hct”) decreases and μ̅ drops significantly when blood flows through small diameter vessels, e.g. <200 μm, which is a property is called both the Fahreus and Fahreus-Lindquist effect. Hematocrit sensitivity on pressure-flow relationships will be tested for a suitable range of flow rates and 10<Hct≦40%. The pressure-flow relationship for gas flow through the adjacent microchannel space will be determined, using the same general approach.

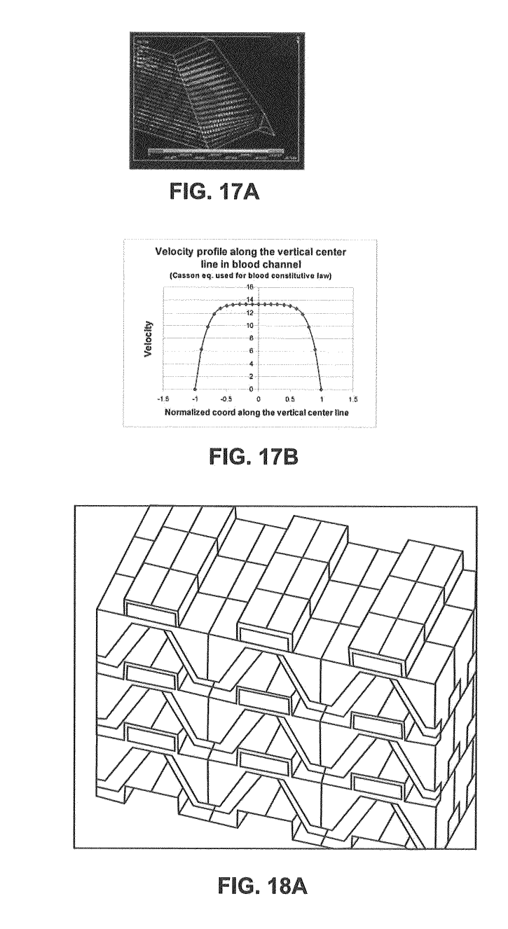

Using Casson's equation for blood, the velocity distribution across the blood channel from which a pressure-flow relationship and the shear stress near the wall is derived, as shown in FIGS. 17A and 17B. FIG. 17A shows the steady state velocity distribution across the blood channel for the roof-top dome channel when perfused under a pressure gradient of 36 μm H₂O. FIG. 17B shows the velocity profile along the vertical center line in blood channel. The Fahreus/Fahreus-Lindquist effects will be included to improve the pressure-flow and shear stress calculations. The influence on the Fahraeus-Lindquist effect of induced red cell shape change in the capillary channels, e.g., from “torpedo” to “parachute”, on the pressure-flow characteristics of the exchangers will be examined.

O₂ Uptake and CO₂ Removal

O₂ and CO₂ exchange will be modeled following the finite element models applied to model the gas exchange in the pulmonary capillary as described in A. O. Frank, C. J. Chuong, R. L. Johnson, J. Appl. Physiol. 82(6): 2036-2044 (1997), herein incorporated by reference. Equivalent permeabilities for O₂ and CO₂ in the polymer-coated nanoporous membranes will be used in the governing diffusion equation. Oxygen transport within red cells should include both diffusion and oxy-hemoglobin reaction kinetics. The gas transport will be modeled when taking discrete red blood cells into consideration. This modeling activity will also examine the influence on the Fahraeus-Lindquist effect of induced red cell shape change in the capillary channels, e.g., from “torpedo” to “parachute”, on the gas exchange characteristics of the exchangers, i.e., oxygen and carbon dioxide fluxes. The model should reveal the progressive changes in mass transport resistance through the cell transit in the micro-channel. The results will be compared with the experimental results for washed red cells suspensions for the “two-stack” exchangers, and thus will be used to refine the membrane design parameters (pore size, pore density distribution, thickness, etc) governing membrane permeability to gases. Overall, balanced O₂ and CO₂ fluxes ensure pH balance and physiological gas exchange levels. Once the model is calibrated, the overall gas transport at blood channel device level in terms of hematocrit (Hct) will be approximated. For a given blood flow rate, with known Hct, the total amount of oxygen uptake per unit time can be calculated from O₂flow^{(plasma & membrane)}.

O₂uptake^{(Ttotal)}=O₂ flow^{(Plasma&membrane)}*(1−Hct)+O₂flow^{(RBC)}*Hct (4)

where O₂flow^{(plasma & membrane)} is the amount of O₂ that diffuses through the membrane when the blood channel contains only plasma, whereas O₂flow^{(RBC)} is the amount of O₂ uptake if the channel is filled (100%) with red cell cytoplasm, including hemoglobin. The plasma-only results can be compared with the experimental gas exchange results using water as the fluid. The major difference between the experimental and theoretical models in this case is the contribution of higher viscosity in the case of the theoretical model, which can be accounted for.

Structural Integrity of the Blood and Gas Channel

Employing measurements of the membrane mechanical properties, the structural integrity and the pressure load of blood and gas channels under prescribed perfusing conditions will be checked. Design modification in pore size, pore density distribution, and thickness, if needed, will be incorporated and implemented in the nanofabrication phase.

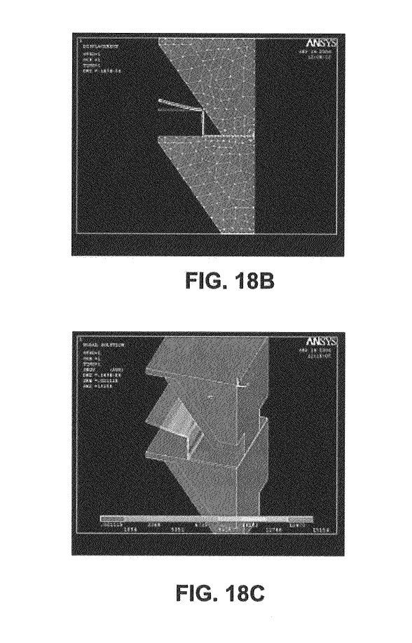

The preliminary analysis reveals the gas exchange membrane deformation observed when micro-channels are loaded with the perfusion pressure distribution corresponding to the desired blood flow rate, as shown in FIG. 18B. FIG. 18A shows the dome channel design with the gas channels connected to gas manifold, which is shown as the yellow rectangles on the side of the dome channel design. The blood channels are shown in red and the gas channels are shown in yellow. Analysis was carried out the dome channel design 200 according to the symmetry and position of the nanoporous membrane. FIG. 18B shows the deflection of the nanoporous membrane under pressure load from blood channel, where displacements are exaggerated to highlight regional differences. FIG. 18C shows the Von Mises stress distribution on the nanoporous membrane due to blood channel pressurization to identify potential weakness in the micro-channel, enabling refinement and improvement.

In one embodiment of the invention, the nanoporous membrane exchanger 100 can be coupled to a miniaturized chandler loop system, employing flowing surrogate fluids and fresh whole blood, for evaluation of oxygen and carbon dioxide exchange efficiency. In another embodiment of the invention, a system for evaluation of the influence of blood proteins, platelets, leukocytes, and red cells on fouling of the exchange surfaces is contemplated. The nanoporous channel 10 O₂ and CO₂ mass transfer coefficients will be determined, employing a “two-stack” nanoporous channel 10. The first assembled SiN/Si-based exchanger unit includes a Chandler flow loop perfused with water at 37° C. Pressure gauges, a rotameter flow meter and temperature controller will be used to characterize pressure-flow-resistance relationships for water and gas flows. Both steady state and pulsatile flows will be studied. Prototype exchanger stacks will be prepared for gas exchange measurements. Short term performance will be studied as a function of channel dimensions, including dimensional creep and liquid and gas operating conditions. The flow loop including the test stack is first primed with degassed water that has been independently brought to a “deoxygenated” (low P_{O2} and high P_{CO2}) state. Gas exchange in this model is measured by delivering oxygen or carbon dioxide mixtures through the gas space, with periodic gas tension microanalysis. O₂ uptake and CO₂ removal will be extracted from the water-based P_{O2} and P_{CO2} and the corresponding mass transfer coefficients determined. Measurements of the two gas exchange rates will be compared with model calculations for model prediction, validation. The O₂ and CO₂ mass transfer coefficients in water will be transformed into corresponding values for flowing blood, as determined for macroscopic nanoporous channels in Eberhart et al. “Mathematical and experimental methods for design and evaluation of membrane oxygenators” Artificial Organs 2:19 (1978), herein incorporated by reference. In addition to the pressure drop and mass transfer measurements in water, SEM of the dissected microchannels after having been perfused with dye solution will be done to identify and examine any water penetration into membrane pores, membrane crack or rupture, and membrane/substrate separation. Both the functional and mechanical integrity of the micro-channel will be ensured.

The two stack microchannel nanoporous membrane exchanger employed in the water experiments will be thoroughly dried, inspected, and will be used as a test-bed for performance with a whole blood surrogate. The surrogate will be washed red cells resuspended in a viscosity and osmolality-matched medium, which is a standard technique in microcirculation research. Single fluid pass O₂ and CO₂ exchange characteristics for the two-stack nanoporous channels by these means, identifying favorable design characteristics, such as channel dimensions, membrane characteristics, RBC suspension, flow rates and pressures gradient). Briefly, O₂ and CO₂ transfer characteristics between blood and gas phase depend on a large number of variables, such as blood and gas flow rates, pressure gradients, temperatures, blood hematocrit, etc. The O₂ transfer rate values are collapsed into a single linear correlation encompassing these parameters. This allows accurate prediction of the critical blood oxygenation rate (rated blood flow), and the entire performance spectrum of the oxygenator on the basis of only two blood inlet property settings preparations. The CO₂ transfer rate analysis involves a more complicated set of experiments owing to the more complex distribution of CO₂ between plasma and cells. Single pass CO₂ experimental analysis can also be performed routinely.

Blood hemolysis rate measurements in the two-stack microchannels can also be performed with RBC suspensions. Centrifugation of test samples, separation of the supernatant and spectrometric measurement of free hemoglobin will be used to determine the hemolysis index, which is a readily performed test in whole blood. The matching of fluid characteristics with whole blood viscosity and osmolality is hypothesized to permit RBC suspension data to serve in lieu of whole blood hemolysis data. Hemolysis index will be evaluated as a function of channel dimensions, membrane characteristics, and fluid pressures and flow rates.

The induction of red cell shape change is maintained in the nanoporous membrane exchanger 100. The nanoporous membrane exchanger 100 includes all classes of mass exchangers used in medical devices, in addition to the oxygenator mass exchangers, that can function with engineered pores in blood channels approximating blood capillary dimensions, such as kidney dialysis & plasmapheresis machines, drug delivery systems, etc.

Further, the nanoporous membrane exchanger 100 also may be used in connection with drug fluid infusion therapies to prevent ischemia and/or to otherwise enhance the effectiveness of the therapies. Examples of drug fluids used in cardiovascular and neurological procedures which may be infused (either before, after or along with the oxygenated blood) in accordance with the present invention include, without limitation, vasodilators (e.g., nitroglycerin and nitroprusside), platelet-actives (e.g., ReoPro and Orbofiban), thrombolytics (e.g., t-PA, streptokinase, and urokinase), antiarrhythmics (e.g., lidocaine, procainamide), beta blockers (e.g., esmolol, inderal), calcium channel blockers (e.g., diltiazem, verapamil), magnesium, inotropic agents (e.g., epinephrine, dopamine), perofluorocarbons (e.g., fluosol), crystalloids (e.g., normal saline, lactated ringers), colloids (albumin, hespan), blood products (packed red blood cells, platelets, whole blood), Na+/H+ exchange inhibitors, free radical scavengers, diuretics (e.g., mannitol), antiseizure drugs (e.g., phenobarbital, valium), and neuroprotectants (e.g., lubeluzole). The drug fluids may be infused either alone or in combination depending upon the circumstances involved in a particular application, and further may be infused with agents other than those specifically listed, such as with adenosine (Adenocard, Adenoscan, Fujisawa), to reduce infarct size or to effect a desired physiologic response.

Additionally, the nanoporous membrane exchanger 100 may be coupled to a heat exchanger to ensure that the temperature of blood remains at 98.6° F. or 37° C. Commercially available, heat exchangers with a large surface area of heat exchange coils or tubing are most efficient in performing the job. However, heat exchangers with large surface areas will inevitably utilize large amounts of prime volume. Therefore, the heat exchanger must be as small as possible to minimize prime volume. A heat exchanger in which the surface area to volume ratio is large will minimize prime volume. Accordingly, the nanoporous membrane exchanger 100 may include a heat exchanger assembly operable to maintain, to increase, or to decrease the temperature of the oxygenated blood as desired in view of the circumstances involved in a particular application. Advantageously, temperatures for the oxygenated blood in the range of about 35° C. to about 37° C. generally will be desired, although blood temperatures outside that range (e.g., perhaps as low as 29° C. or more) may be more advantageous provided that patient core temperature remains at safe levels in view of the circumstances involved in the particular application. Temperature monitoring may occur, e.g., with one or more thermocouples, thermistors or temperature sensors integrated into the electronic circuitry of a feedback controlled system, so that an operator may input a desired perfusate temperature with an expected system response time of seconds or minutes depending upon infusion flow rates and other parameters associated with the active infusion of cooled oxygenated blood.

The nanoporous membrane exchanger 100 may also be operatively coupled to a pump assembly for pumping blood to the blood channels. The blood pump assembly may be one of the many commercially available and clinically accepted blood pumps suitable for use with human patients. One example of such a pump is the Model 6501 RFL3.5 Pemco peristaltic pump available from Pemco Medical, Cleveland, Ohio. The blood to be oxygenated comprises blood withdrawn from the patient, so that the blood pump assembly includes a blood inlet disposed along a portion of a catheter or other similar device at least partially removably insertable within the patient's body; a pump loop that in combination with the catheter or other device defines a continuous fluid pathway between the blood inlet and the membrane oxygenator assembly; and a blood pump for controlling the flow of blood through the pump loop, i.e., the flow of blood provided to the membrane oxygenator assembly.

Additionally, the nanoporous membrane exchanger 100 may be coupled to an oxygen supply assembly for supplying a regulated source of oxygen to the gas channels of the nanoporous membrane exchanger. The oxygen supply assembly comprises an apparatus including a chamber coupled to a regulated source of oxygen gas that maintains a desired pressure in the chamber. A physiologic fluid (e.g., saline) enters the chamber through a nozzle. The nozzle forms fluid droplets into which oxygen diffuses as the droplets travel within the chamber. The nozzle comprises an atomizer nozzle adapted to form a droplet cone definable by an included angle alpha., which is about 20 to about 40 degrees at operating chamber pressures (e.g., about 600 p.s.i.) for a pressure drop across the nozzle of greater than approximately 15 p.s.i. The nozzle is a simplex-type, swirled pressurized atomizer nozzle including a fluid orifice of about 100 μm diameter. The nozzle forms fine fluid droplets of less than about 100 μm diameter and of about 25μ. The fluid advantageously is provided to the chamber by a pump operatively coupled to a fluid supply assembly. The fluid is provided at a controlled rate based on the desired oxygen-supersaturated fluid outlet flow rate. At the bottom of the chamber, fluid collects to form a pool which includes fluid having a dissolved gas volume normalized to standard temperature and pressure of between about 0.5 and about 3 times the volume of the solvent. The fluid is removed from the chamber via a pump, which permits control of the flow rate, or by virtue of the pressure in the chamber for delivery to a given location, e.g., to a blood oxygenation assembly.

Alternatively, the nanoporous membrane exchanger 100 is coupled with an oxygen-supersaturated fluid to the gas channels. Exemplary apparatus and methods for the preparation and delivery of oxygen-supersaturated fluids are disclosed in U.S. Pat. No. 5,407,426, U.S. Pat. No. 5,569,180, U.S. Pat. No. 5,599,296 and U.S. Pat. No. 5,893,838 each of which is incorporated herein by reference.

The nanoporous membrane exchanger 100 may include one or more gas bubble detectors operatively coupled to the blood channels, at least one of which is capable of detecting the presence of microbubbles, e.g., bubbles with diameters of about 100 μm to about 1000 μm. In addition, the nanoporous membrane exchanger may include one or more macrobubble detectors to detect larger bubbles, such as bubbles with diameters of about 1000 μm or more. Such macrobubble detectors may comprise any suitable commercially available detector, such as an outside, tube-mounted bubble detector including two transducers measuring attenuation of a sound pulse traveling from one side of the tube to the other. One such suitable detector may be purchased from Transonic Inc. of New York. The microbubble and macrobubble detectors provide the physician or caregiver with a warning of potential clinically significant bubble generation. Such warnings also may be obtained through the use of transthoracic 2-D echo (e.g., to look for echo brightening of myocardial tissue) and the monitoring of other patient data. The bubble detection system is able to discriminate between various size bubbles. Further, the bubble detection system advantageously operates continuously and is operatively coupled to the overall system so that an overall system shutdown occurs upon the sensing of a macrobubble.

The nanoporous membrane exchanger 100 also may include various conventional items, such as sensors, flow meters (which also may serve a dual role as a macrobubble detector), or other clinical parameter monitoring devices; hydraulic components such as accumulators and valves for managing flow dynamics; access ports which permit withdrawal of fluids; filters or other safety devices to help ensure sterility; or other devices that generally may assist in controlling the flow of one or more of the fluids in the system. Any such devices are positioned within the exchanger and used so as to avoid causing the formation of clinically significant bubbles within the fluid flow paths, and/or to prevent fluid flow disruptions, e.g., blockages of capillaries or other fluid pathways. Further, the exchanger comprises a biocompatible system acceptable for clinical use with human patients.

The nanoporous membrane exchanger may also be coupled to a carbon dioxide removal unit for removing the carbon dioxide in the gas channels after the gas has exchanged carbon dioxide with the blood channels.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A nanoporous membrane exchanger comprising:
a. at least two nanoporous channels, wherein the nanoporous channels include at least one gas channel, at least one blood channel, and at least one nanoporous membrane communicating between the gas channel and the blood channel.

2. The nanoporous membrane exchanger of claim 1, wherein the nanoporous membrane comprises silicon and an array of nanopores.

3. The nanoporous membrane exchanger of claim 2, wherein the nanopores include an average nanopore diameter in a range of 50 to 500 nanometers.

4. The nanoporous membrane exchanger of claim 3, wherein the nanoporous membrane includes a blood compatible coating and a perfluorinated monomer coating.

5. The nanoporous membrane exchanger of claim 4, wherein the nanoporous channels are bonded together with a biocompatible bonding material.

6. The nanoporous membrane oxygenator of claim 5, wherein the blood channel includes a surface area to blood volume ratio in the range of 0.0065 to 0.168 μm⁻¹.

7. The nanoporous membrane exchanger of claim 5, wherein the blood channel and the gas channel include a blood gas volume ratio in the range of 15 to 156%.

8. The nanoporous membrane exchanger of claim 7, further comprising a plurality of gas channels in operable communication by transport processes with the blood channel.

9. The nanoporous membrane exchanger of claim 8, wherein the membrane includes a thickness in the range of 700 to 1100 nm.

10. The nanoporous membrane exchanger of claim 9, wherein the nanoporous channels include a thickness in the range of 30 to 50 μm.

11. The nanoporous membrane exchanger of claim 10, wherein the membrane includes a Young's modulus in the range of 0.3×10⁷ to 0.3×10⁸ N/mm².

12. The nanoporous membrane exchanger of claim 4, wherein the nanopores include a deposited polymer film to regulate the gas permeation rates of the nanoporous membrane.

13. A method for making a nanoporous membrane exchanger, comprising the steps:
a. depositing silicon nitride onto a silicon layer;
b. anisotropically etching along the <100> direction of the silicon layer;
c. etching in the <111> direction to create a membrane;
d. drilling the membrane to create a plurality of nanopores; and
e. micromachining a gas channel and a blood channel, wherein the plurality of nanopores communicate with the gas channel and the blood channel.

14. The method of claim 13, wherein the drilling step further comprises focused ion beam drilling with fluorine gas.

15. The method of claim 14, wherein the focused ion beam drilling step further comprises coating the membrane with a metal.

16. The method of claim, 13, further comprising coating the nanoporous membrane and nanopores by variable duty cycle pulse plasma deposition of a polymer.

17. The method of claim 16, wherein the etching in the <111> direction further comprises doping a layer with boron atoms to define a base of the blood channel.

18. The method of claim 17, further comprising forming a gas channel in communication with the membrane and the blood channel.

19. The method of claim 18, further comprising bonding a first gas channel and a first blood channel with a second gas channel and a second blood channel.

20. The method of claim 16, wherein the coating step further comprises depositing perfluorinated monomers.

21. The method of claim 13, wherein the drilling step comprises nanoimprinting the nanopores on the membrane.

22. A method of performing mass exchange comprising:
a. introducing blood into at least one blood channel in a nanoporous channel, wherein the nanoporous channel includes at least one gas channel and a nanoporous membrane;
b. introducing gas into the gas channel in the nanoporous channel to subject the blood flowing through the blood channel to mass exchange; and
c. discharging the blood which has been subjected to the mass exchange from the nanoporous channel

23. The method of claim 22, further comprising removing bubbles in the blood in the nanoporous channel after the blood has been subjected to the gas exchange and before the blood is discharged from the nanoporous channel.

24. The method according to claim 22, further comprising passing the blood through a heat exchanger.
